SEQUENCE LISTING (18 sequences)

SEQ ID NO: 1 - DNA, 28 bp, Artificial Sequence
  forward primer h_Trip10_promoter_clo_F2
ggcctcaggttaaagtttgaccctagga

SEQ ID NO: 2 - DNA, 23 bp, Artificial Sequence
  reverse primer h_Trip10_promoter_clo_R1
ccttctgcccgcctcattcgcaa

SEQ ID NO: 3 - DNA, 26 bp, Artificial Sequence
  forward primer Trip_TR_1_F
gaagtttatctgggagtctcagcacg

SEQ ID NO: 4 - DNA, 29 bp, Artificial Sequence
  reverse primer Trip_TR_1_R
catgccaatacaatgtaggctgctctaca

SEQ ID NO: 5 - DNA, 27 bp, Artificial Sequence
  forward primer Trip_TR_2_F
cactgcattctagttgtggtttgtcca

SEQ ID NO: 6 - DNA, 25 bp, Artificial Sequence
  reverse primer Trip_TR_2_R
caacagatggctggcaactagaagg

SEQ ID NO: 7 - DNA, 27 bp, Artificial Sequence
  forward primer To_EGFP_F1
gaggtctatataagcagagctctccct

SEQ ID NO: 8 - DNA, 24 bp, Artificial Sequence
  reverse primer To_EGFP_R1
ccttgctcaccatggtggcgaccg

SEQ ID NO: 9 - DNA, 25 bp, Artificial Sequence
  forward primer To_EGFP_F2
atcacatggtcctgctggagttcgt

SEQ ID NO: 10 - DNA, 28 bp, Artificial Sequence
  reverse primer To_EGFP_R2
gcaaacaacagatggctggcaactagaa

SEQ ID NO: 11 - DNA, 20 bp, Artificial Sequence
  primer h_Trip10_L_M1
gagaggacgagagggatttc

SEQ ID NO: 12 - DNA, 22 bp, Artificial Sequence
  primer h_Trip10_R_M1
ccaaacactacgattcgaaaac

SEQ ID NO: 13 - DNA, 27 bp, Artificial Sequence
  primer h_Trip10_L_M2
gaagtttatttgggagttttagtacgt

SEQ ID NO: 14 - DNA, 20 bp, Artificial Sequence
  primer h_Trip10_R_M2
aataacctctctcaaccgcc

SEQ ID NO: 15 - DNA, 28 bp, Artificial Sequence
  primer BR_137
tctaacaattataaactccaaccaccaa

SEQ ID NO: 16 - DNA, 25 bp, Artificial Sequence
  primer BR_138
gggaagatgggatagaagggaatat

SEQ ID NO: 17 - DNA, 23 bp, Artificial Sequence
  forward primer h_bis_Trip10_TR_F2
gggaaaggggaaaaggagatggg

SEQ ID NO: 18 - DNA, 29 bp, Artificial Sequence
  reverse primer h_bis_Trip10_TR_R2
atcttaccaactttccccttctaaaaaac

(19) United States
(12) Patent Application Publication
(10) Pub. No.: US 2011/0091875 A1
(43) Pub. Date: Apr. 21, 2011

(54) THREE-COMPONENT GENE EXPRESSION REPORTING SYSTEM FOR MAMMALIAN CELLS AND APPLICATIONS OF THE SAME

(75) Inventors: Yu-Wei Leu, Changhua City (TW); Shu-Huei Hsiao, She-Tou Township (TW); Chia-Chen Hsu, South District (TW); Tim Hui-Ming Huang, Columbus, OH (US)

(73) Assignee: National Chung Cheng University, Ming-Hsiung Township (TW)

(21) Appl. No.: 12/702,797

(22) Filed: Feb. 9, 2010

(30) Foreign Application Priority Data
Oct. 16, 2009 (TW) 098135114

(51) Int. Cl. C12Q 1/68; C12N 15/63; C12N 5/00

(52) U.S. Cl. 435/6; 435/320.1; 435/325

(57) ABSTRACT

Disclosed herein is a three-component gene expression reporting system for mammalian cells, the system including a first expression cassette, a second expression cassette, and a methylated polynucleotide. The three-component gene expression reporting system can be used to establish recombinant mammalian cells for use in the screening of a demethylating agent.

### CROSS-REFERENCE TO RELATED APPLICATION

This application claims priority of Taiwanese Application No. 098135114, filed on Oct. 16, 2009.

### BACKGROUND OF THE INVENTION

1. Field of the Invention

This invention relates to a three-component gene expression reporting system for mammalian cells, which comprises a first expression cassette, a second expression cassette, and a methylated polynucleotide. The three-component gene expression reporting system can be used to establish recombinant mammalian cells for screening demethylating agents.

2. Description of the Related Art

The epigenetic regulation, found to be essential for all the cellular functions, includes a number of processes that modify DNA and histone structures, such as DNA methylation, histone modification and remodeling, as well as gene silencing by small RNAs (H. S. Cho et al. (2007), Journal of Biochemistry and Molecular Biology, 40:151-155). Accumulating evidence has shown that epigenetic modifications have a crucial role in pathological disorders, including, for example, cancer, inherited diseases, and chronic inflammatory diseases.

DNA methylation, e.g., the addition of a methyl group from S-adenosyl-L-methionine (SAM) to the fifth carbon position of the cytosine residue in a CpG dinucleotide, is effected by the catalytic action of DNA methyltransferase (DNMT). The CpG dinucleotides are usually distributed within stretches of 1- to 2-kb GC-rich DNA, named CpG islands, located in the promoter and/or first exon of 60% of human genes (A. Bird (2002), Genes Dev., 16:6-21; M. Ehrlich (1982), Nucleic Acids Res., 10:2709-2721).

Promoter methylation is known to participate in reorganizing chromatin structure and also plays a role in transcriptional inactivation (A. Bird (2002), supra; M. Ehrlich (2003), J. Cell Biochem., 88:899-910). Studies have suggested that the CpG island in an active promoter is usually non-methylated, with the surrounding chromatin displaying an “open” configuration, allowing for the access of transcription factors and other co-activators to initiate gene expression (E. Ballestar and M. Esteller (2002), Carcinogenesis, 23:1103-1109; P. A. Jones and S. B. Baylin (2002), Nat. Rev. Genet., 3:415-428; K. P. Nephew and Tim H-M Huang (2003), Cancer Lett., 190:125-133). Furthermore, transcription factor occupancy may make the promoter inaccessible to repressors or other chromatin remodeling proteins. In contrast, the CpG islands in an inactive promoter may become methylated, with the associated chromatin exhibiting a “closed” configuration. As a result, the methylated area is no longer accessible to transcription factors, thereby disabling the functional activity of the promoter (P. A. Jones and S. B. Baylin (2002), supra; S. B. Baylin et al. (2001), Hum. Mol. Genet., 10:687-692; M. R. Rountree et al. (2001), Oncogene, 20:3156-3165).

DNA methylation is a very stable epigenetic modification. This modification is material for the mammalian development, the establishment and maintenance of imprinted genes, and the stability of genome in cells. Abnormal DNA methylations, independent of introduction or removal of methyl groups, not only lead to the incidence of disease but are also involved in the carcinogenesis of cells. Abnormal DNA methylations are proposed to cause cancer in two major ways, one being the hypermethylation in certain genes especially the tumor suppressor genes, and the other being the global DNA demethylation.

Previous studies have shown that DNA methylation participates in the transcription repression of genes (J. Boyes and A. Bird (1991), Cell, the 64(6):1123-1134; J. Boyes and A. Bird (1992), EMBO J., 11(1):327-333; S. U. Kass et al., (1997), Curr Biol, 7(3): p. 157-165; X. Nan et al., (1997), Cell, 88(4):471-481; R. Singal and G. D. Ginder (1999), Blood, 93(12):4059-4070).

Although DNA methylation is considered to be a rather stable epigenetic modification, more evidences reveal that dynamic DNA methylation can be observed in certain genes that participate in differentiation and development, or in the downstream genes of certain signaling pathways (e.g., the estrogen receptor signaling pathway), and in genes that affect the carcinogenesis of cells.

For the treatment of diseases like cancer, chemical demethylating agents or DNMT inhibitors are proposed to restore the expression of tumor suppressor genes. An example of the demethylating agents that have been approved by the U.S. Food and Drug Administration (FDA) and widely used in clinics is decitabine (5-aza-2′-deoxycytidine, trade name Dacogen), which is used to treat myelodysplastic syndrome by repressing DNMT. The success in DNA demethylation and restoration of respective gene expression indicate that the epigenetic regulation like DNA methylation is more reversible and feasible than the genetic modification/therapy.

In the past two decades, in order to understand the DNA methylation pattern of a gene of interest, there have been developed several analytical methods, including e.g., the bisulfite genomic sequencing method (Marianne Frommer et al. (1992), Proc. Natl. Acad. Sci., USA, 89:1827-1831), the methylation-specific polymerase chain reaction (MSP) (James G. Herman et al. (1996), Proc. Natl. Acad. Sci. USA, 93:9821-9826), the combined bisulfite restriction analysis (COBRA) (Zhenggang Xiong and Peter W. Laird (1997), Nucleic Acids Research, 25:2532-2534), the restriction landmark genomic scanning (RLGS) (Joseph F. Costello et al. (2000), Nature Genetics, 25:132-138), the CpG island microarrays (Pearlly S. Yan et al. (2001), Cancer Research, 61:8375-8380), and the Methyl-DNA ImmunoPrecipitation (MeDIP) (Filipe V. Jacinto et al. (2008), BioTechniques, 44:35-43). However, in these analytical methods, genomic DNA is required to serve as a sample source for the analysis of DNA methylation pattern, and destruction of cells is inevitable in the harvesting process of genomic DNA. Therefore, when these analytical methods are used to screen a demethylating agent, they fail to enable continuous observation of the tested cells for a long period of time, nor can they continuously and immediately show a dynamic change of the DNA methylation patterns within the tested cells before, during and after the administration of the demethylating agent.

The reporter gene system is a common tool widely employed in the study of regulatory promoter and enhancer sequences and in the investigation of transcription factors, in which a regulatory sequence of interest is combined with a selected reporter construct, followed by subjecting to analysis along with the transcription factors associated therewith. If a proper reporter gene system is selected, the expression level of the reporter gene will be correlated to the transcriptional activity of the promoter, enhancer or transcription factor of interest. In order to ensure this correlation, the reporter gene should not disturb a transformed cell's metabolism, and nor should it be endogenously expressed in a target cell capable of generating background signals. Exemplary examples of the reporter gene systems known in the art include, but are not limited to: chloramphenicol acetyl Transferase (CAT) reporter gene system, lacZ reporter gene system, β-glucuronidase (GUS) reporter gene system, green fluorescent protein (GFP) reporter gene system, etc. However, when applied in a promoter assay, the reporter gene systems existing in the art can only be used to analyze an interested promoter's transcriptional activity in promoting the expression of downstream gene(s) via detecting the expression level of the reporter gene, and are unable to determine the epigenetic regulation patterns (in particular the DNA methylation pattern) occurring on the interested promoter, as well as the influence of the epigenetic regulation upon the interested promoter's transcriptional activity.

In view of the reporter gene system's characteristic that an interested promoter's transcriptional activity can be determined via detecting a reporter gene's expression level, it is highly desirable to develop a new analytical method that is based on the reporter gene system and that can display the DNA methylation pattern of an interested promoter dynamically.

Therefore, a goal intended to be achieved for a new gene expression reporting system is the ability to instantaneously observe the change of DNA methylation pattern (i.e., increased or decreased DNA methylations) of a specific promoter in a cell at a specific stage. Further, since DNA methylation participates in the gene silencing regulation, the new gene expression reporting system should meet the following two prerequisites: enabling the instantaneous observation of living cells for a long time, and allowing the replay of the intracellular epigenetic modifications in regulating the gene transcription phenomena. Therefore, based on these two prerequisites, the applicants attempted to design a gene expression reporting system that enables the instantaneous observation of the dynamic DNA methylation phenomenon of a specific promoter in a living cell.

### SUMMARY OF THE INVENTION

Therefore, according to a first aspect, this invention provides a three-component gene expression reporting system for mammalian cells, the system comprising:

a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene;

a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and

a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   wherein introduction of the methylated polynucleotide into a mammalian cell that has been co-transfected by the first and second expression cassettes results in the methylation of the one or more CpG islands of the second promoter sequence in the co-transfected mammalian cell and progeny cell thereof, thereby repressing the first nucleic acid sequence to express the first gene product.

According to a second aspect, this invention provides a kit for screening a demethylating agent, the kit comprising:

(a) a recombinant mammalian cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and

(b) a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   wherein introduction of the methylated polynucleotide into the recombinant mammalian cell results in the methylation of the one or more CpG islands of the second promoter sequence in the recombinant mammalian cell and progeny cell thereof, thereby repressing the first nucleic acid sequence to express the first gene product.

According to a third aspect, this invention provides a method for screening a candidate compound as a demethylating agent, the method comprising:

providing a first population of a recombinant mammalian cell, each cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product;

introducing into the first population of the recombinant mammalian cell a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   so that the one or more CpG islands of the second promoter sequence in the first population of the recombinant mammalian cell or progeny cell thereof become methylated, thereby repressing the first nucleic acid sequence to express the first gene product;

cultivating the first population of the recombinant mammalian cell for a period of time to obtain a second population of the recombinant mammalian cell;

detecting the second population of the recombinant mammalian cell to obtain a first expression level of the reporter gene;

treating the second population of the recombinant mammalian cell with a candidate compound, followed by cultivation for a period of time, so as to obtain a third population of the recombinant mammalian cell; and
detecting the third population of the recombinant mammalian cell to obtain a second expression level of the reporter gene,
   wherein the candidate compound is deemed as a demethylating agent if the obtained second expression level of the reporter gene is lower than the obtained first expression level of the reporter gene.

According to a fourth aspect, this invention provides a recombinant mammalian cell comprising:

a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and

a second expression cassette, which comprises a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product,

wherein in the recombinant mammalian cell, the one or more CpG islands in the second promoter sequence have been methylated to result in repression of the first nucleic acid sequence, thereby allowing the reporter gene to be expressed in the recombinant mammalian cell.

According to a fifth aspect, this invention provides a method for screening a candidate compound as a demethylating agent, the method comprising:

providing a first population of a recombinant mammalian cell as described in the fourth aspect;

detecting the first population of the recombinant mammalian cell to obtain a first expression level of the reporter gene;

treating the first population of the recombinant mammalian cell with a candidate compound, followed by cultivation for a period of time, so as to obtain a second population of the recombinant mammalian cell; and

detecting the second population of the recombinant mammalian cell to obtain a second expression level of the reporter gene,

wherein the candidate compound is deemed as a demethylating agent if the obtained second expression level of the reporter gene is lower than the obtained first expression level of the reporter gene.

### BRIEF DESCRIPTION OF THE DRAWINGS

The above and other features and advantages of the present invention will become apparent in the following detailed description of the preferred embodiment with reference to the accompanying drawing, of which:

FIG. 1 schematically shows the practice of a preferred embodiment of the three-component gene expression reporting system for mammalian cells according to this invention, in which a first expression cassette and a second expression cassette are schematically shown to be located on two different vectors, where the first expression cassette comprises in sequence along a transcription direction: a CMV promoter (the block marked P_{CMV}), a TATA box (the block marked TATA), two tetracycline operators (the two blocks marked TetO₂), and an EGFP gene (the block marked EGFP), as well as a hygromycin resistance gene (the block marked Hygro) located upstream of the CMV promoter and acting as a marker gene for the first expression cassette; and the second expression cassette comprises a Trip10 promoter (the block marked Trip10 promoter), and a nucleic acid sequence (the block marked Tet Repressor) located downstream of the Trip10 promoter and encoding a tetracycline repressor (represented by the white circles), as well as a neomycin resistance gene (the block marked Neo) located upstream of the Trip10 promoter and acting as a marker gene for the second expression cassette. The white head match-shaped objects vertically disposed on the Trip10 promoter block indicate the locations of non-methylated CpG islands in the Trip10 promoter, whereas the black head match-shaped objects vertically disposed on the Trip10 promoter block indicate the locations of methylated CpG islands in the Trip10 promoter. The bent arrow represents the transcription initiation site, and the symbol “x” represents the blocking of transcription. The comb tooth-shaped objects with black head match-shaped objects disposed thereon represent methylated single-stranded polynucleotides, and the star-shaped object represents EGFP;

FIG. 2 shows the construction scheme of recombinant plasmid pTrip10-TR, in which P_{CMV} represents a CMV promoter; TetR represents a tetracycline repressor gene; Amp represent an ampicillin resistance gene; Neo represents a neomycin resistance gene; SV40-A represents a polyadenylation signal of SV40; SV40-P represents a SV40 promoter; and EcoRI, XbaI and Bg/II represent the cutting sites of the corresponding restriction enzymes, respectively;

FIG. 3 shows the construction of recombinant plasmid pTetO-EGFP, in which P_{CMV} represents a CMV promoter; 2× TetO₂ represents two tetracycline operators; EGFP represents an enhanced green fluorescent protein encoding gene; Hygro represents a hydromycin resistance gene; and XhoI represents the cutting site of the corresponding restriction enzyme;

FIG. 4 shows the relative locations of the regions (defined by pairs of triangles) that correspond to the nucleotide sequences as amplified from four primer pairs (TR1, TR2, EGFP1 and EGFP2) designed based on the nucleotide sequences of the recombinant plasmids pTrip10-TR and pTetO-EGFP, respectively, and the relative location of the region that corresponds to the nucleotide sequence as amplified from a primer pair (Bis) used in a bisulfite sequencing experiment, in which P_{Trip10} represents a Trip10 promoter; TetR represents a tetracycline repressor gene; Neo represents a neomycin resistance gene; P_{CMV} represents a CMV promoter; 2× TetO₂ represents two tetracycline operators; EGFP represents an enhanced green fluorescent protein encoding gene; polyA represents a polyadenylation signal; Hygro represents a hydromycin resistance gene; EcoRI, XbaI, Bg/II and XhoI represent the cutting sites of the corresponding restriction enzymes, respectively; Bis represents a Bis primer pair; TR1 represents a TR1 primer pair; TR2 represents a TR2 primer pair; EGFP1 represents an EGFP1 primer pair; and EGFP2 represents an EGFP2 primer pair;

FIG. 5 shows the agarose gel electrophoresis results of the PCR products as obtained from PCR experiments using the genomic DNA of SC6 and SC9 cells as templates and four different primer pairs (TR1, TR2, EGFP1 and EGFP2) designed based on the nucleotide sequences of recombinant plasmids pTrip10-TR and pTetO-EGFP, in which the blank group used water to run the PCR experiment; the positive control group used a mixture of recombinant plasmids pTrip10-TR and pTetO-EGFP to run the PCR experiment; and the control group used the genomic DNA of MCF7 cells to run the PCR experiment;

FIG. 6 shows the expression of EGFP in SC6 cells as cultivated in the screening medium with (+) or without (−) the addition of 1 μg/mL doxycycline (400× magnification);

FIG. 7 shows the expression of EGFP in SC6 cells having been transfected with single-stranded me_Trip10 DNA as observed at different time points (0 hr, 24 hrs, 72 hrs and 120 hrs) under visible light (the four upper panels) or at a wavelength of 507 nm (the four lower panels) (400× magnification);

FIG. 8 is a bar diagram showing the EGFP intensity percentages of the observed EGFP expressions in the four lower panels of FIG. 7 as quantitatively analyzed by an image analyzing software;

FIG. 9 is a bar diagram that shows the methylation percentages of the Trip10 promoter and the first exon region in SC6 cells having been transfected with single-stranded me_Trip10 DNA, in which the methylation percentages were measured by the semi-quantitative methylation-specific PCR (qMSP); the negative control group used the genomic DNA (bisulfite converted) of MCF7 cells to run the qMSP; and the mock-transfection control group used the genomic DNA (bisulfite converted) of SC6 cells having been transfected with a transfecting mixture containing a non-methylated DNA carrying the human Trip10 promoter to run the qMSP;

FIG. 10 shows the bisulfite sequencing results of the Trip 10 promoter and the first exon region in SC6 cells having been transfected with single-stranded me_Trip10 DNA, in which the mock-transfection control group used the genomic DNA of SC6 cells having been transfected with a non-methylated DNA carrying the human Trip10 promoter to implement the bisulfite sequencing experiment; TetR represents a tetracycline repressor gene; the black and white circles represent methylated and non-methylated CpG dinucleotides, respectively; and the bent arrow represents the transcription initiation site;

FIG. 11 shows the expression of EGFP in SC6 cells having been transfected with single-stranded me_Trip10 DNA as observed at different time points (0 hr, 24 hrs, 72 hrs and 120 hrs) under visible light (the four upper panels) or at a wavelength of 507 nm (the four lower panels) (400× magnification), wherein the SC6 cells were cultivated in medium containing 17β-estradiol before, during and after transfection with the single-stranded me_Trip10 DNA;

FIG. 12 is a bar diagram showing the EGFP intensity percentages of the observed EGFP expressions in the four lower panels of FIG. 11 as quantitatively analyzed by an image analyzing software;

FIG. 13 shows the expression of EGFP in SC6 cells having been transfected with single-stranded me_Trip10 DNA as observed at different time points (2 days, 4 days, 6 days and 8 days) under visible light (the four upper panels) or at a wavelength of 507 nm (the four lower panels) (400× magnification), wherein the SC6 cells were cultivated in medium containing 5-aza-2′-deoxycytidine, with (+) or without (−) addition of 178-estradiol, after transfection with the single-stranded me_Trip10 DNA; and

FIG. 14 is a bar diagram showing the EGFP intensity percentages of the observed EGFP expressions in the four lower panels of FIG. 13 as quantitatively analyzed by an image analyzing software.

### DETAILED DESCRIPTION OF THE INVENTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described. For clarity, the following definitions are used herein.

As used herein, the term “gene” refers to a DNA sequence, including but not limited to a DNA sequence that can be transcribed into mRNA which can be translated into polypeptide chains, transcribed into rRNA or tRNA, or serve as recognition sites for enzymes and other proteins involved in DNA replication, transcription and regulation. This definition includes various sequence polymorphisms, mutations, and/or sequence variants wherein such alterations do not affect the function of the gene product. The term “gene” is intended to include not only regions encoding gene products but also regulatory regions including, e.g., promoters, termination regions, translational regulatory sequences (such as ribosome binding sites and internal ribosome entry sites), enhancers, silencers, insulators, boundary elements, replication origins, matrix attachment sites, and locus control regions. The term “gene” further includes all introns and other DNA sequences spliced from the mRNA transcript, along with variants resulting from alternative splice sites. The term “gene” includes, but is not limited to, structural genes, immunity genes and secretory (transport) genes.

As used herein, the term “reporter gene” refers to a polynucleotide encoding a molecule that can be easily directly detected or that can be easily identified through characteristics of host cells. Exemplary reporter genes encode enzymes (e.g., β-galactosidase (lacZ), chloramphenicol acetyltransferase (CAT), β-glucuronidase (GUS)), luminescent or fluorescent proteins (e.g., green fluorescent protein (GFP) and variants thereof), antigenic epitopes (e.g., Histidine-tag or influenza hemagluttinin tag), mRNA of distinct sequences, and the like.

As used herein, the term “gene expression reporting system” refers to any system, device or tool that comprises a reporter gene for detecting gene expression.

As used herein, the term “expression cassette” refers to a construct of genetic material that contains coding sequences and enough regulatory information to direct proper transcription and translation of the coding sequences in a recipient cell. The expression cassette may be inserted into a vector for targeting to a desired host cell and/or into a subject.

As used herein, the term “vector” refers to any carrier of exogenous DNA that is useful for transferring the DNA to a host cell for replication and/or appropriate expression of the exogenous DNA by the host cell. The term “vector” includes cloning and expression vehicles, such as plasmids, phages, transposons, cosmids, liposomes, virus-liposome complexes, DNA-viral conjugates, RNA/DNA oligonucleotides, viruses, bacteria, etc.

As used herein, the term “promoter” can be used interchangeably with the term “promoter sequence” and refers to a DNA regulatory region capable of binding RNA polymerase in a cell and initiating transcription of a downstream (3′ direction) coding sequence. The promoter is bound at its 3′ terminus by the translation start codon of a coding sequence and extends upstream (5′ direction) to include a minimum number of bases or elements necessary to initiate transcription. Promoters which cause a gene to be expressed in most cell types at most times are commonly referred to as “constitutive promoters”. Promoters which cause conditional expression of a structural nucleotide sequence under the influence of changing environmental conditions or developmental conditions are commonly referred to as “inducible promoter.”

As used herein, the term “operator” can be used interchangeably with the term “operator region” and refers to the region of DNA that is upstream (5′) from a gene(s) and to which one or more regulatory proteins (repressor or activator) bind to control the expression of the gene(s).

As used herein, the term “upstream” and “downstream” refer to the position of an element of nucleotide sequence. “Upstream” signifies an element that is more 5′ than the reference element. “Downstream” signifies an element that is more 3′ than the reference element.

As used herein, the term “polynucleotide” refers to a sequence of nucleotides connected by phosphodiester linkages. A polynucleotide of this invention can be a deoxyribonucleic acid (DNA) molecule or ribonucleic acid (RNA) molecule in either single- or double-stranded form. Nucleotide bases are indicated herein by a single letter code: adenine (A), guanine (G), thymine (T), cytosine (C), inosine (I) and uracil (U). A polynucleotide of this invention can be prepared using standard techniques well known to one of ordinary skill in the art. This term is not to be construed as limiting with respect to the length of a polymer, and encompasses known analogues of natural nucleotides, as well as nucleotides that are modified in the sugar and/or phosphate moieties. This term also encompasses nucleic acids containing modified backbone residues or linkages, which are synthetic, naturally occurring, and non-naturally occurring, which have similar binding properties as the reference nucleic acid, and which are metabolized in a manner similar to the reference nucleotides.

As used herein, the term “identical to” refers to two or more nucleotide sequences that are the same when compared and aligned for maximum correspondence, as measured using sequence comparison algorithms or by inspection.

As used herein, the term “complementary to” refers to the ability of two nucleotide sequences to bind sequence-specifically to each other by hydrogen bonding through their purine and/or pyrimidine bases according to the usual Watson-Crick rules for forming duplex nucleic acid complexes.

As used herein, the term “gene product” refers primarily to proteins and polypeptides encoded by a nucleic acid, but further encompasses nucleic acids encoded by other nucleic acids (e.g., non-coding and regulatory RNAs such as tRNA, sNRPs).

The conventional analytical methods for the analysis of DNA methylation pattern require genomic DNA to serve as a sample source. However, since destruction of cells is inevitable in the harvesting process of genomic DNA, in the preliminary in vitro screening of a demethylating agent, the conventional analytical methods fail to enable continuous observation of the tested cells during the screening procedure, nor can they continuously and immediately show a dynamic change of the DNA methylation patterns within the tested cells before, during and after the administration of the demethylating agent.

On the other hand, the reporter gene systems existing in the art can only be used to analyze an interested promoter's transcriptional activity in promoting the expression of downstream gene(s) via detecting the expression level of the reporter gene, and are unable to determine the DNA methylation pattern occurring on the interested promoter, as well as the influence of the DNA methylation pattern upon the interested promoter's transcriptional activity. If a new reporting system that is capable of dynamically reflecting the DNA methylation pattern of a promoter can be developed based on the conventional reporter gene systems, said new reporting system is believed to have a great potential for use in the field of new drug research and development.

Furthermore, since DNA methylation participates in the gene silencing regulation, the new gene expression reporting system should meet the following two prerequisites: enabling the instantaneous observation of living cells for a long time, and allowing the replay of the intracellular epigenetic modifications in regulating the gene transcription phenomena. Therefore, based on these two prerequisites, the applicants endeavored to develop a gene expression reporting system that enables the instantaneous observation of the dynamic DNA methylation phenomenon of a specific promoter in a living cell.

Accordingly, this invention provides a three-component gene expression reporting system for mammalian cells, the system comprising:

a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene;

a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and

a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   wherein introduction of the methylated polynucleotide into a mammalian cell that has been co-transfected by the first and second expression cassettes results in the methylation of the one or more CpG islands of the second promoter sequence in the co-transfected mammalian cell and progeny cell thereof, thereby repressing the first nucleic acid sequence to express the first gene product.

As used herein, the term “transcription direction” refers to the direction of 5′ to 3′ addition of nucleotides to nascent RNA transcripts.

As used herein, the term “mammalian cell” include cells that are derived from a normal tissue or a tumor of a mammal. According to this invention, the mammal may be selected from the group consisting of humans, bovine, sheep, goats, horses, dogs, cats, rabbits, rats, and mice.

Preferably, the mammalian cell is a human cell. In a preferred embodiment of this invention, the mammalian cell is a normal human cell line. In another preferred embodiment of this invention, the mammalian cell is a human tumor/cancer cell line. In a more preferred embodiment of this invention, the mammalian cell is a human breast cancer cell line selected from the group consisting of MCF7, ZR-75-1, T-47-D, BT-474, MDA-MB-134 and MDA-MB-361.

According to this invention, a promoter that is able to initiate the expression of the reporter gene in a selected mammalian cell can be used as the first promoter sequence. According to this invention, the first promoter sequence may be a natural promoter from genes of viruses, bacteriophages, prokaryotic cells or eukaryotic cells, or may be an artificially synthetic promoter made by modifying the aforementioned natural promoter. According to this invention, the first promoter sequence is preferably a constitutive promoter.

According to this invention, the first promoter sequence comprises a promoter selected from the group consisting of a CMV promoter, a SV40 initial promoter, a RSV-promoter, a HSV-TK promoter, a U6 promoter, a CMV-HSV thymidine kinase promoter, a SRα promoter, and a HIV•LTR promoter. In a preferred embodiment of this invention, the first promoter sequence comprises a CMV promoter.

According to this invention, in order to prevent interference of exogenous transcription factors, both the first operator region and the first nucleic acid sequence are preferably heterologous to the mammalian cell. As used herein, the term “heterologous to the genome of the host cell” means that the polynucleotide does not naturally occur in the genome of the host cell.

According to this invention, both the first operator region and the first nucleic acid sequence are preferably derived from a gene of a microbial cell. For instance, both the first operator region and the first nucleic acid sequence may be derived from a gene of any one of the following: a virus, a bacterial cell, a yeast cell, a fungal cell, and an algal cell. More preferably, both the first operator region and the first nucleic acid sequence are derived from a gene of a bacterial cell such as an Escherichia coli cell.

In a preferred embodiment of this invention, the first operator region comprises a tetracycline operator, and the first gene product encoded by the first nucleic acid sequence is a tetracycline repressor.

In another preferred embodiment of this invention, the first operator region comprises a Lac operator, and the first gene product encoded by the first nucleic acid sequence is a Lac repressor.

In yet another preferred embodiment of this invention, the first operator region comprises a GUS operator, and the first gene product encoded by the first nucleic acid sequence is a GUS repressor.

According to this invention, the reporter gene encodes a reporter gene product selected from the group consisting of: green fluorescent protein (GFP), enhanced green fluorescent protein (EGFP), red fluorescent protein, yellow fluorescent protein, blue fluorescent protein, td-Tomato, mCherry, firefly luciferase, Renilla luciferase, β-galactosidase, β-glucuronidase, and a fusion protein comprising one or more of the aforementioned proteins. In a preferred embodiment of this invention, the reporter gene product is EGFP.

According to this invention, the second promoter sequence is in an actively transcribed state in a selected mammalian cell. Namely, the second promoter sequence is in an unmethylated state.

According to this invention, the second promoter sequence may comprise a promoter and a first exon both of which come from an identical gene. As used herein, the term “first exon” refers to the first region of a gene that encodes a polypeptide or a polypeptide region and is located downstream of the promoter region of the gene.

Preferably, the second promoter sequence comprises a promoter selected from the group consisting of a Trip10 promoter, a Casp8AP2 promoter, an ENSA promoter, and a H1C1 promoter. In an embodiment of this invention, the second promoter sequence comprises a Trip10 promoter and the 1^{st} to 339^{th} nucleotide residues of the first exon of the Trip10 gene.

According to this invention, the methylated polynucleotide may have a length ranging from 22 to 2,000 nucleotides or even longer. Preferably, the methylated polynucleotide has a length ranging from 60 to 1,500 nucleotides. More preferably, the methylated polynucleotide has a length ranging from 150 to 1000 nucleotides. In a preferred embodiment of this invention, the methylated polynucleotide has a length of around 500 nucleotides.

As used herein, the term “transfection” can be used interchangeably with the term “transformation” and refers to the introduction of an exogenous nucleic acid molecule into a selected host cell. According to techniques known in the art, an exogenous nucleic acid molecule (e.g., a recombinant DNA construct or a recombinant vector) can be introduced into a competent host cell by various techniques, such as gene gun, electroporation, microinjection, heat shock, calcium phosphate precipitation, magnetofection, nucleofection, lipofection, use of transfection reagents, use of cationic polymers, etc.

As used herein, the term “co-transfected” and the term “co-transfection” refer to the introduction of two or more identical or different nucleic acid sequences into a selected recipient cell.

According to this invention, the first expression cassette further comprises a first marker gene, and the second expression cassette further comprises a second marker gene. The first marker gene, the second marker gene, and the reporter gene differ from each other, so that a mammalian cell co-transfected by the first and second expression cassettes can be selected from a screening test.

As used herein, the term “marker gene” refers to any transcribable polynucleotide molecule whose expression can be screened for or scored in some way. Methods of testing for marker gene expression are known to those skilled in the art.

Preferably, the first and second marker genes suitable for use in this invention are independently selected from the group consisting of a hygromycin resistance gene, a neomycin resistance gene, a gentamycin resistance gene, a blasticidin resistance gene, a zeocin resistance gene, and a puromycin resistance gene. In a preferred embodiment of this invention, the first marker gene is a hygromycin resistance gene, and the second marker gene is a neomycin resistance gene.

According to this invention, the first and second expression cassettes may be present in different vectors. Alternatively, the first and second expression cassettes may be present in the same vector and spaced apart from each other. The vector may be an expression system in linear or circular form, and covers expression systems that remain episomal or integrate into the host cell genome. The expression system may or may not have the ability to self-replicate, and it may drive only transient expression in a host cell.

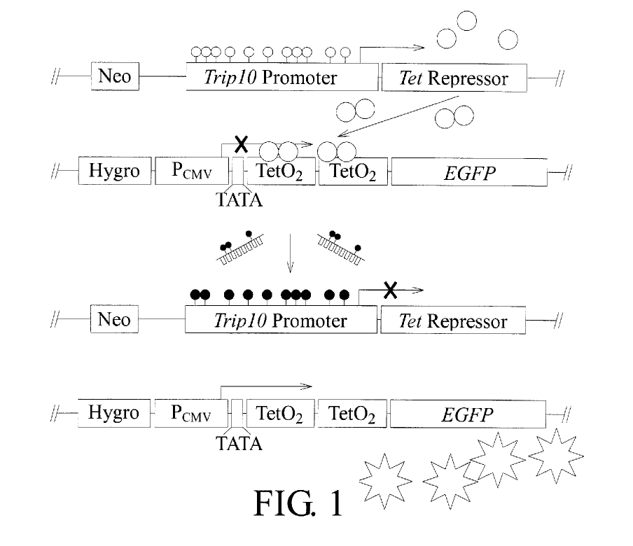

FIG. 1 schematically shows the practice of a preferred embodiment of the three-component gene expression reporting system for mammalian cells according to this invention, in which a first expression cassette and a second expression cassette are designed to be located on two different vectors. The first expression cassette comprises in sequence along a transcription direction: a CMV promoter, a TATA box, two tetracycline operators (2× TetO₂), and an EGFP gene, as well as a hygromycin resistance gene located upstream of the CMV promoter and acting as a marker gene for the first expression cassette. The second expression cassette comprises a Trip10 promoter, and a nucleic acid sequence located downstream of the Trip10 promoter and encoding a tetracycline repressor (TetR, represented by the white circles), as well as a neomycin resistance gene located upstream of the Trip10 promoter and acting as a marker gene for the second expression cassette.

The first and second expression cassettes can be co-transfected into a mammalian cell. Referring to FIG. 1, when the CpG islands in the sequence of the Trip10 promoter are non-methylated (the white head match-shaped objects vertically disposed on the Trip10 promoter block indicate the locations of non-methylated CpG islands), the Trip10 promoter is able to initiate the expression of the nucleic acid sequence that encodes TetR, and the expressed TetR will form a dimeric molecule that can bind to the TetO₂ in the first expression cassette. Therefore, expression of the EGFP gene in the co-transfected mammalian cell is repressed due to the binding of the TetR dimer to the TetO₂.

Referring to FIG. 1, when the methylated single-stranded polynucleotides (represented by the comb tooth-shaped objects with black head match-shaped objects disposed thereon) are introduced into the co-transfected mammalian cell, the CpG islands of the Trip10 promoter in the co-transfected mammalian cell and progeny cell thereof will become methylated (the black head match-shaped objects vertically disposed on the Trip10 promoter block indicate the locations of methylated CpG islands), thus rendering the Trip10 promoter unable to initiate the nucleic acid sequence to express TetR. Due to the effect of cell mitosis, the TetO₂ in the co-transfected mammalian cell as well as progeny cell thereof becomes free of binding by the TetR dimer so that expression of the EGFP gene occurs in the co-transfected mammalian cell and progeny cell thereof (the star-shaped object represents the expressed EGFP).

The applicants found that when using the methylated single-stranded polynucleotide to treat the co-transfected mammalian cell, one is able to instantaneously and continuously observe the dynamic methylation of the Trip10 promoter in living cell.

The applicants further found that if the co-transfected mammalian cell was treated with a demethylating agent subsequent to the treatment with the methylated single-stranded polynucleotide, the CpG islands of the Trip10 promoter in the co-transfected mammalian cell and progeny cell thereof will become demethylated, thus allowing the Trip10 promoter to initiate the nucleic acid sequence to express TetR. As a consequence, expression of the EGFP gene in the co-transfected mammalian cell and progeny cell thereof is repressed again.

The applicants further found that when a demethylating agent is used to treat the co-transfected mammalian cell that has been treated with the methylated single-stranded polynucleotide, a reagent that can assist in reversion of the methylation of the one or more CpG islands of the Trip10 promoter can be simultaneously applied to the co-transfected mammalian cell, so as to accelerate the demethylation of the Trip10 promoter. The reagent may be an estrogen or an estrogen analogue such as 17β-estradiol.

Therefore, the three-component gene expression reporting system of this invention can be used to establish a recombinant mammalian cell for instantaneous observation of the dynamic methylation of a specific promoter in a living cell. The three-component gene expression system and the recombinant mammalian cell according to this invention are expected to have great potential for use in the screening of demethylating agents.

Accordingly, this invention provides a kit for screening a demethylating agent, the kit comprising:

(a) a recombinant mammalian cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and

(b) a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   wherein introduction of the methylated polynucleotide into the recombinant mammalian cell results in the methylation of the one or more CpG islands of the second promoter sequence in the recombinant mammalian cell and progeny cell thereof, thereby repressing the first nucleic acid sequence to express the first gene product.

According to this invention, the recombinant mammalian cell is obtained by using the first and second expression cassettes to co-transfect a cell selected from the group consisting of a human cell, a bovine cell, a sheep cell, a goat cell, a horse cell, a dog cell, a cat cell, a rabbit cell, a rat cell, and a mouse cell. Preferably, the recombinant mammalian cell is obtained by co-transfecting a human cell with the first and second expression cassettes. In a preferred embodiment of this invention, the human cell is a normal human cell line. In another preferred embodiment of this invention, the human cell is a human tumor/cancer cell line. In a more preferred embodiment of this invention, the mammalian cell is a human breast cancer cell line selected from the group consisting of: MCF7, ZR-75-1, T-47-D, BT-474, MDA-MB-134, and MDA-MB-361.

This invention also provides a method for screening a candidate compound as a demethylating agent, the method comprising:

providing a first population of a recombinant mammalian cell, each cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product;

introducing into the first population of the recombinant mammalian cell a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
   so that the one or more CpG islands of the second promoter sequence in the first population of the recombinant mammalian cell or progeny cell thereof become methylated, thereby repressing the first nucleic acid sequence to express the first gene product;

cultivating the first population of the recombinant mammalian cell for a period of time to obtain a second population of the recombinant mammalian cell;

detecting the second population of the recombinant mammalian cell to obtain a first expression level of the reporter gene;

treating the second population of the recombinant mammalian cell with a candidate compound, followed by cultivation for a period of time, so as to obtain a third population of the recombinant mammalian cell; and

detecting the third population of the recombinant mammalian cell to obtain a second expression level of the reporter gene,

wherein the candidate compound is deemed as a demethylating agent if the obtained second expression level of the reporter gene is lower than the obtained first expression level of the reporter gene.

Concerning the detection of the recombinant mammalian cell so as to obtain the expression level of the reporter gene, this can be made by implementing the methodologies that are well known to and commonly used by those skilled in the art. For example, detecting the recombinant mammalian cell may be implemented by colormetry, fluorimetry, luminescent analysis, enzyme-linked immunosorbent assay (ELISA), or flow cytometry. In a preferred embodiment of this invention, detecting the recombinant mammalian cell is implemented by fluorimetry.

According to this invention, when using the candidate compound to treat the second population of the recombinant mammalian cell, a reagent that can assist in reversion of the methylation of the one or more CpG islands of the second promoter sequence may be simultaneously applied to the second population of the recombinant mammalian cell. In a preferred embodiment of this invention, the second promoter sequence comprises a Trip10 promoter, and the reagent is estrogen. In another preferred embodiment of this invention, the second promoter sequence comprises a Trip10 promoter, and the reagent is an estrogen analogue such as 17β-estradiol.

This invention also provides a recombinant mammalian cell comprising:

a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and

a second expression cassette, which comprises a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product,

wherein in the recombinant mammalian cell, the one or more CpG islands in the second promoter sequence have been methylated to result in repression of the first nucleic acid sequence, thereby allowing the reporter gene to be expressed in the recombinant mammalian cell.

According to this invention, the recombinant mammalian cell is obtained by using the first and second expression cassettes to co-transfect a cell selected from the group consisting of a human cell, a bovine cell, a sheep cell, a goat cell, a horse cell, a dog cell, a cat cell, a rabbit cell, a rat cell, and a mouse cell, followed by introducing into the co-transfected cell thus obtained a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii).

Preferably, the recombinant mammalian cell is obtained by using the first and second expression cassettes to co-transfect a human cell, followed by introducing the methylated polynucleotide into the co-transfected human cell thus obtained.

This invention further provides a method for screening a candidate compound as a demethylating agent in which a recombinant mammalian cell as described above is used, the method comprising:

providing a first population of the recombinant mammalian cell as described above;

detecting the first population of the recombinant mammalian cell to obtain a first expression level of the reporter gene;

treating the first population of the recombinant mammalian cell with a candidate compound, followed by cultivation for a period of time, so as to obtain a second population of the recombinant mammalian cell; and

detecting the second population of the recombinant mammalian cell to obtain a second expression level of the reporter gene,

wherein the candidate compound is deemed as a demethylating agent if the obtained second expression level of the reporter gene is lower than the obtained first expression level of the reporter gene.

According to this invention, when the candidate compound is used to treat the first population of the recombinant mammalian cell, a reagent that can assist in reversion of the methylation of the one or more CpG islands of the second promoter sequence may be simultaneously applied to the first population of the recombinant mammalian cell. In a preferred embodiment of this invention, the second promoter sequence comprises a Trip10 promoter; and the reagent is an estrogen. In another preferred embodiment of this invention, the second promoter sequence comprises a Trip10 promoter, and the reagent is an estrogen analogue such as 17β-estradiol.

This invention will be further described by way of the following examples. However, it should be understood that the following examples are solely intended for the purpose of illustration and should not be construed as limiting the invention in practice.

### EXAMPLES

### Experimental Materials

### 1. Growth Medium:

Unless otherwise indicated, in connection with the growth medium used in the following examples for cultivating mammalian cells, the minimum essential medium (MEM, Gibco BRL) was selected to serve as the basal medium and was supplemented with 10% fetal bovine serum (FBS) (Biological Industries), 2 mM glutamine (Gibco BRL), and 100 mg/mL penicillin/streptomycin.

### 2. Screening Medium:

The screening medium used in the following examples was generally formulated with the materials as described in the preceding section, entitled “1. Growth medium,” and was further supplemented with 0.5 mg/mL G418 and 0.4 mg/mL hygromycin.

3. Human Breast Cancer Cell Line MCF7 that Expresses Estrogen Receptor α⁺ (ERα⁺):

MCF7 cells [ATCC accession no. HTB-22] were placed in a flask containing the growth medium and cultivated in an incubator with culture conditions set at 37° C. and 95% O₂/5% CO₂. Medium changes were performed twice a week. Cell passages were performed when the cell density reached 90% confluence.

### General Experimental Procedures:

### 1. Transformation:

A selected plasmid was evenly mixed with competent E. coli DH5α cells (Yeastern Biotech, Cat. No. YE607-J), and the resultant mixture was placed on ice for 5 minutes. The resultant mixture was subsequently spread onto solid agar plates containing 50 μg/mL ampicillin, followed by cultivation at 37° C. for 16 hours. Thereafter, ampicillin-resistant colonies were picked up from the solid agar plates and then inoculated into LB broth containing 50 μg/mL ampicillin, followed by cultivation at 37° C. for 16 hours.

### 2. Transfection:

Each well of 6-well plates was plated with cells for conducting transfection later (1×10⁵ cells/2 mL growth medium/well). After cell attachment, the liquid in each well of the 6-well plates was removed, and a transfection mixture containing 300 μL MEM, 6 μL at DMRIE-C reagent (Invitrogen) and a selected DNA for transfecting the cells was subsequently added, followed by cultivation for 4 hours. Thereafter, each well of the 6-well plates was added with the growth medium until a final liquid volume of 1 mL in each well was reached, and cultivation of the cells was continued for further 48 hours.

### Example 1

### Construction of Recombinant Plasmid pTrip10-Tr Containing a Human Trip10 Promoter and a TetR Gene

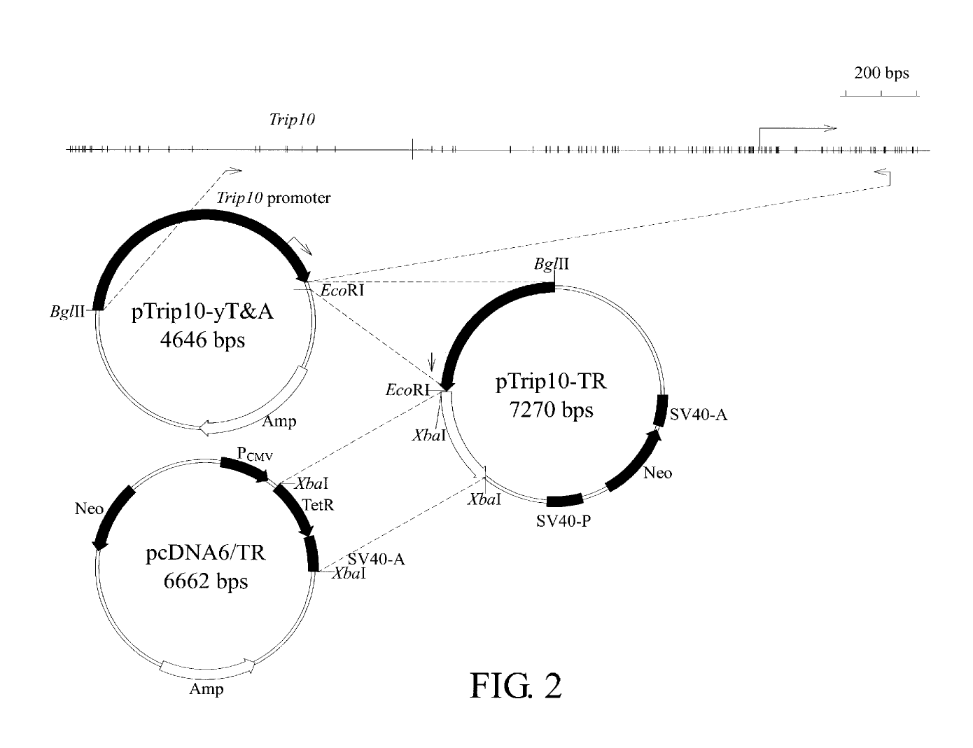

A recombinant plasmid pTrip10-TR containing a human Trip10 promoter and a tetracycline repressor encoding gene (TetR) was obtained according to the construction scheme shown in FIG. 2 and the procedures described below.

1. Construction of Recombinant Plasmid pTrip10-yT&A

Based on a reference sequence deposited in the NCBI database under accession number NM_{—}004240 (Homo sapiens thyroid hormone receptor interactor 10 (Homo sapiens Trip10), mRNA) and the nucleotide sequence of human Gene TRIP10 deposited at the UCSC website under accession number UCSC ID uc002mfs. 1, the following primer pair were designed:

h_Trip10_promoter_clo_F2 primer:

| | |
|---|---|
| 5′-ggcctcaggttaaagtttgaccctagga-3′ | (SEQ ID NO: 1) |
| h_Trip10_promoter_clo_R1 primer: | |
| 5′-ccttctgcccgcctcattcgcaa-3′ | (SEQ ID NO: 2) |

The genomic DNA of human epithelial ovarian cancer cell line CP70 (kindly afforded by Dr. Robert Brown of University of Glasgow) as purified by a Genomic DNA Purification Kit (GeneMark, Cat. No. DP02-150) was used as a template in a polymerase chain reaction (PCR) experiment using the h_Trip10_promoter_clo primer pair described above and having the reaction conditions as shown in Table 1, such that a PCR product (1916 bps) containing the human Trip10 promoter was obtained, wherein the PCR product had a nucleotide sequence substantially corresponding to a nucleotide sequence spanning from the upstream 1,577th nucleotide residue of the human Trip10 gene to the 339th nucleotide residue of the first exon of the human Trip10 gene.

TABLE 1
Reaction conditions used in the PCR experiment

| Contents | Volume (μL) |
|---|---|
| Genomic DNA of CP70 cells (0.01 μg/μL) | 2 |
| h_Trip10_promoter_clo_F2 primer (2.5 μM) | 1 |
| h_Trip10_promoter_clo_R1 primer (2.5 μM) | 1 |
| dNTPs (10 mM) | 1 |
| Taq DNA polymerase | 0.2 |
| 10X Taq buffer solution containing (NH₄)₂SO₄ | 2.5 |
| MgCl₂ (25 mM) | 5 |
| ddH₂O | 12.3 |

Operation conditions: Denaturation at 95° C. for 5 minutes, followed by 37 cycles of the following reactions: denaturation at 95° C. for 30 seconds, primer annealing at 60° C. for 30 seconds, and elongation at 72° C. for 2 minutes; and finally extension at 72° C. for 4 minutes.

The amplified PCR products as obtained from the PCR experiment were verified by 1% agarose gel electrophoresis, followed by purification and recovery using a Clean and Gel Extraction Kit (BioKit, Cat. No. Bio-C300). Finally, the recovered PCR product was dissolved in 20 μL ddH₂O to form a PCR product solution.

6 μL of the PCR product solution was evenly admixed with 1 μL of T₄ DNA ligase, 1 μL ligation buffer A, 1 μL ligation buffer B and 1 μL of yT&A cloning vector (2728 bps, containing ampicillin resistance gene therein), these four reagents being included in the yT&A Cloning Vector Kit (Yeastern Biotech, Cat. No. YC001), and the resultant mixture was placed at 4° C. for 16 hours to allow completion of the ligation reaction, such that a recombinant plasmid pTrip10-yT&A with a construction as shown in FIG. 2 was obtained.

Thereafter, E. coli DH5α cells were transformed with the recombinant plasmid pTrip10-yT&A and cultivated according to the procedures as described in the preceding section, entitled “1. Transformation,” of the General Experimental Procedures. The resultant culture was harvested and the recombinant plasmid pTrip10-yT&A contained therein was extracted using a High-Speed Plasmid Mini Kit (Geneaid), followed by cleavage using restriction enzyme HindIII. The resultant cleavage products were subjected to agarose gel electrophoresis so as to verify the molecular weight of the recombinant plasmid pTrip10-yT&A. The verified plasmid was subjected to a sequencing analysis conducted by Tri-I Biotech, Inc.

2. Construction of Recombinant Plasmid pcDNA3-Trip10

Plasmid pcDNA3 (5446 bps, including a CMV promoter (P_{CMV}), a neomycin resistance gene, a Bg/II restriction site, and an EcoRI restriction site) (Invitrogen) was cleaved with restriction enzymes Bg/II and EcoRI so that a cleavage product (926 bps) with P_{CMV} and a cleavage product (4520 bps) not including P_{CMV} were obtained. The cleavage product without P_{CMV} was subsequently purified and recovered via agarose gel electrophoresis, followed by extraction using the Clean and Gel Extraction Kit.

In the meantime, the recombinant plasmid pTrip10-yT&A as obtained above was cleaved with restriction enzymes Bg/II and EcoRI, such that a DNA fragment (1928 bps) containing the human Trip10 promoter was obtained. The DNA fragment containing the human Trip10 promoter was subsequently purified and recovered via agarose gel electrophoresis, followed by extraction using the Clean and Gel Extraction Kit.

10 μL of the DNA fragment containing the human Trip10 promoter (0.07 μg/μL in ddH₂O) was evenly admixed with 4 μL of the cleavage product without P_{CMV} (0.02 μg/μL, in ddH₂O), 2 μL of T₄ DNA ligase, 2 μL of 10× ligation buffer, and 2 μL of 50% polyethylene glycol 4000 solution (50% PEG4000 solution), the latter three reagents being obtained from Fermentas (Cat. No. EL0334). The resultant mixture was placed at 4° C. for 16 hours to allow completion of ligation reaction, so that a recombinant plasmid pcDNA3-Trip10 was obtained (not shown in FIG. 2).

E. coli DH5α cells were transformed with the recombinant plasmid pcDNA3-Trip10 and cultivated according to the procedures as described in the preceding section, entitled “1. Transformation,” of the General Experimental Procedures. The resultant culture was harvested and the recombinant plasmid pcDNA3-Trip10 contained therein was extracted using a High-Speed Plasmid Mini Kit (Geneaid), followed by restriction enzyme cleavage to verify the molecular weight thereof. The verified recombinant plasmid pcDNA3-Trip10 was subjected to a sequencing analysis conducted by Tri-I Biotech, Inc.

3. Construction of Recombinant Plasmid pTrip10-TR

The recombinant plasmid pcDNA3-Trip10 as obtained above was cleaved with restriction enzyme XbaI, followed by admixing with 5 μL of calf Intestinal alkaline phosphatase (CIAP, Promega) (0.01 U/μL) and 5 μL of 10× CIAP Reaction Buffer (Promega). After even mixing, the resultant mixture was added with ddH₂O until a volume of 50 μL was reached, followed by placing at 37° C. for 30 minutes so as to prevent the XbaI-cleaved linear recombinant plasmid pcDNA3-Trip10 from self-ligation.

In the meantime, plasmid pcDNA6/TR (6662 bps, including a TetR gene) (Invitrogen, Cat. No. V1025-20) was cleaved with restriction enzyme XbaI so as to obtain a DNA fragment (821 bps) with the TetR gene. The DNA fragment thus obtained was subsequently purified and recovered via agarose gel electrophoresis, followed by extraction using the Clean and Gel Extraction Kit.

4 μL of the CIAP-treated XbaI-cleaved recombinant plasmid pcDNA3-Trip10 (0.1 μg/μL, in ddH₂O) was admixed with 20 μL of the DNA fragment with the TetR gene (0.15 μg/μL, in ddH₂O), followed by addition of 4 μL of T₄ DNA ligase, 4 μL of 10× ligation buffer, and 4 μL of 50% PEG4000 solution. After even mixing, the resultant mixture was placed at 4° C. for 16 hours to allow completion of ligation reaction, such that a recombinant plasmid pTrip10-TR (7270 bps) having a construction as shown in FIG. 2 was obtained.

E. coli DH5α cells were transformed with the recombinant plasmid pTrip10-TR and cultivated according to the procedures as described in the preceding section, entitled “1. Transformation,” of the General Experimental Procedures. The resultant culture was harvested and the recombinant plasmid pTrip10-TR contained therein was extracted using a High-Speed Plasmid Mini Kit (Geneaid), followed by restriction enzyme cleavage to verify the molecular weight thereof. The verified recombinant plasmid pTrip10-TR was subjected to a sequencing analysis conducted by Tri-I Biotech, Inc.

### Example 2

### Construction of Recombinant Plasmid pTetO-EGFP Containing an EGFP Gene and Tetracycline Operators

A recombinant plasmid pTetO-EGFP comprising two tetracycline operators (2× TetO₂) and an enhanced green fluorescent protein (EGFP) gene capable of being regulated by the 2× TetO₂ was constructed as follows:

First, plasmid pEGFP-C1 (4731 bps, including a G418 resistance gene) (Clontech, Cat. No. 6084-1) was cleaved with restriction enzyme NheI, followed by treatment with T₄ DNA polymerase (New England BioLabs), so that the cleavage product's sticky ends were converted to blunt ends. After recovery, the resultant blunt-ended cleavage product was cleaved with restriction enzyme XhoI to yield a DNA fragment (752 bps) with the EGFP gene and a DNA fragment (3983 bps) without the EGFP gene. The DNA fragment with the EGFP gene was subsequently purified and recovered via agarose gel electrophoresis, followed by extraction using the Clean and Gel Extraction Kit.

In the meantime, pcDNA5/TO [5667 bps, containing two tetracycline operators (2× TetO₂) and a hygromycin resistance gene] (Invitrogen, Cat. No. V1033-20) was cleaved with restriction enzyme HindIII, followed by treatment with T₄ DNA polymerase, so that the cleavage product's sticky ends were converted to blunt ends. After recovery, the resultant blunt-ended cleavage product was cleaved with restriction enzyme XhoI to yield a small DNA fragment (74 bps) and a large DNA fragment (5597 bps). The large DNA fragment was subsequently purified and recovered via agarose gel electrophoresis, followed by extraction using the Clean and Gel Extraction Kit.

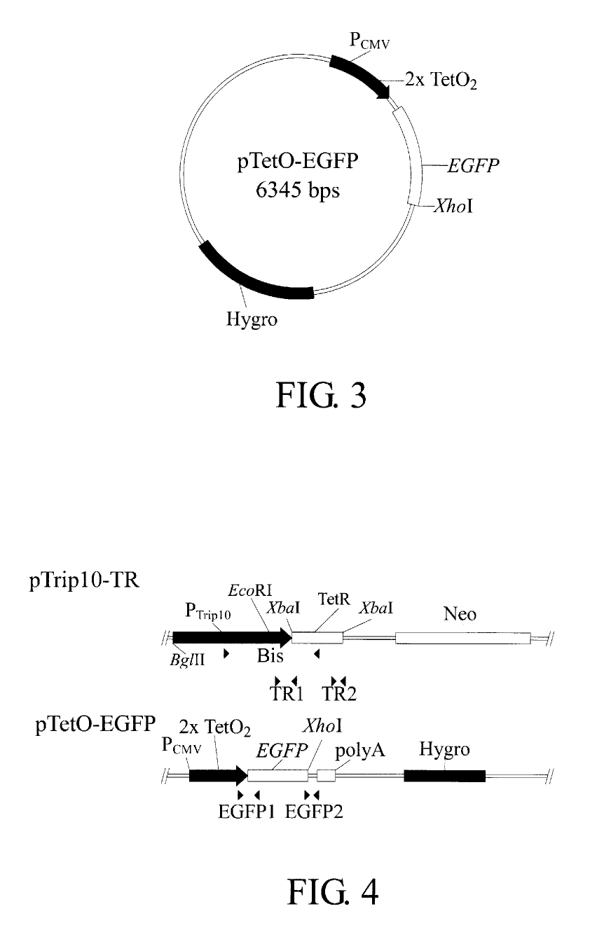

12 μL of the DNA fragment with EGFP gene (0.08 μg/μL, in ddH₂O) was admixed with 2 μL of the large DNA fragment (0.02 μg/μL, in ddH₂O), followed by addition of 2 μL of 1⁻⁴ DNA ligase, 2 μL of 10× ligation buffer, and 2 μL of 50% PEG4000 solution. After even mixing, the resultant mixture was placed at 4° C. for 16 hours to allow completion of ligation reaction, such that a recombinant plasmid pTetO-EGFP (6345 bps) having a construction as shown in FIG. 3 was obtained.

Subsequently, E. coli DH5α cells were transformed with the recombinant plasmid pTetO-EGFP and cultivated according to the procedures as described in the preceding section, entitled “1. Transformation,” of the General Experimental procedures. The resultant culture was harvested and the recombinant plasmid pTetO-EGFP contained therein was extracted using a High-Speed Plasmid Mini Kit, followed by restriction enzyme cleavage to verify the molecular weight thereof. The verified recombinant plasmid pTetO-EGFP was subjected to a sequencing analysis conducted by Tri-I Biotech, Inc., and was confirmed to have a construction as shown in FIG. 3.

### Example 3

### Transfection of Human Breast Cancer Cell Line MCF7 with Recombinant Plasmids pTrip10-TR and pTetO-EGFP

### Experimental Procedure:

MCF7 cells were plated into each well of 6-well plates (1×10⁵ cells/2 mL growth medium/well) and then transfected using 9 μL of a solution containing the recombinant plasmid pTrip10-TR (1.12 μg/μL, dissolved in ddH₂O) as obtained from the above Example 1 and 1 μL of a solution containing the recombinant plasmid pTetO-EGFP (1 μg/μL, dissolved in ddH₂O) as obtained from the above Example 2 according to the procedures as set forth in the preceding section, entitled “2. Transfection,” of the General Experimental Procedures.

After removal of liquid from each well, the transfection-treated MCF7 cells (at a cell density of 20/mL) were transferred to 10-cm Petri dishes containing screening medium for cultivation. About 1˜2 weeks later, the transfection-treated MCF7 cells that were able to grow in the presence of G418 and hygromycin were picked up and further cultivated in the screening medium. Cell passages were performed so as to expand the cell number of the transfection-treated MCF7.

In order to verify that the recombinant plasmids pTrip10-TR and pTetO-EGFP had been incorporated into the genomic DNA of the transfection-treated MCF7 cells to result in the formation of stable clone (SC), the following experiment was conducted.

First, two clones of the transfection-treated MCF7 cells, which were designated as SC6 and SC9, respectively were subjected to genomic DNA extraction using the Genomic DNA Purification Kit. The genomic DNAs thus obtained were used as templates in a PCR experiment using four specific primer pairs as shown in Table 2 and having the reaction conditions as shown in Table 3. These four primer pairs were designed based on the nucleotide sequences of the recombinant plasmids pTrip10-TR and pTetO-EGFP, and the relative locations of the regions (defined by pairs of triangles) that correspond to the nucleotide sequences as amplified from these four primer pairs are shown in FIG. 4.

In the meantime, same PCR experiments using water (the blank group), a mixture of recombinant plasmids pTrip10-TR and pTetO-EGFP (positive control group) and the genomic DNA of MCF7 cells (control group), respectively, were concomitantly performed.

TABLE 2
Primer pairs used in the PCR experiment

| Primer Pair | Primer | Sequence (5′→3′) |
|---|---|---|
| TR1 | Trip_TR_1_F | gaagtttatctgggagtctcagcacg |
| | | (SEQ ID NO: 3) |
| | Trip_TR_1_R | catgccaatacaatgtaggctgctct |
| | | aca |
| | | (SEQ ID NO: 4) |
| TR2 | Trip_TR_2_F | cactgcattctagttgtggtttgtc |
| | | ca |
| | | (SEQ ID NO: 5) |
| | Trip_TR_2_R | caacagatggctggcaactagaagg |
| | | (SEQ ID NO: 6) |
| EGFP1 | To_EGFP_F1 | gaggtctatataagcagagctctc |
| | | cct |
| | | (SEQ ID NO: 7) |
| | To_EGFP_R1 | ccttgctcaccatggtggcgaccg |
| | | (SEQ ID NO: 8) |
| EGFP2 | To_EGFP_F2 | atcacatggtcctgctggagttcgt |
| | | (SEQ ID NO: 9) |
| | To_EGFP_R2 | gcaaacaacagatggctggcaacta |
| | | gaa |
| | | (SEQ ID NO: 10) |

TABLE 3
Reaction conditions of PCR experiment

| Contents | Volume (μL) |
|---|---|
| Genomic DNA of SC6 cell/genomic DNA of SC9 cell | 2 |
| (0.01 μg/μL) | |
| Forward primer (2.5 μM) | 1 |
| Reverse primer (2.5 μM) | 1 |
| dNTPs (10 mM) | 1 |
| Taq DNA polymerase | 0.2 |
| 10X Taq buffer solution containing (NH₄)₂SO₄ | 2.5 |
| MgCl₂ (25 mM) | 5 |
| ddH₂O | 12.3 |

Operation conditions: Denaturation at 95° C. for 5 minutes, followed by 37 cycles of the following reactions: denaturation at 95° C. for 30 seconds, primer annealing at 60° C. for 30 seconds, and elongation at 72° C. for 2 minutes; and finally extra extension at 72° C. for 4 minutes.

The PCR products as obtained from the PCR experiments were subjected to agarose gel electrophoresis for analysis and verification.

On the other hand, the stable clones could be verified by the use of doxycycline, which is a tetracycline analogue capable of binding to TetR to cause the repression of 2× TetO₂ by TetR to be removed. Briefly, the transfection-treated MCF7 cells as selected from the screening medium were subjected to cultivation in a screening medium containing 1 μg/mL doxycycline at 37° C. for 16-24 hours. The expression of EGFP in the cultivated cells was observed using an inverted fluorescence microscope (Nikon) at a wavelength of 507 nm and under 400× magnification, and photographs were recorded using a digital camera connected to the inverted fluorescence microscope. The transfection-treated MCF7 cells cultivated in the absence of doxycycline were used as a control group.

### Results:

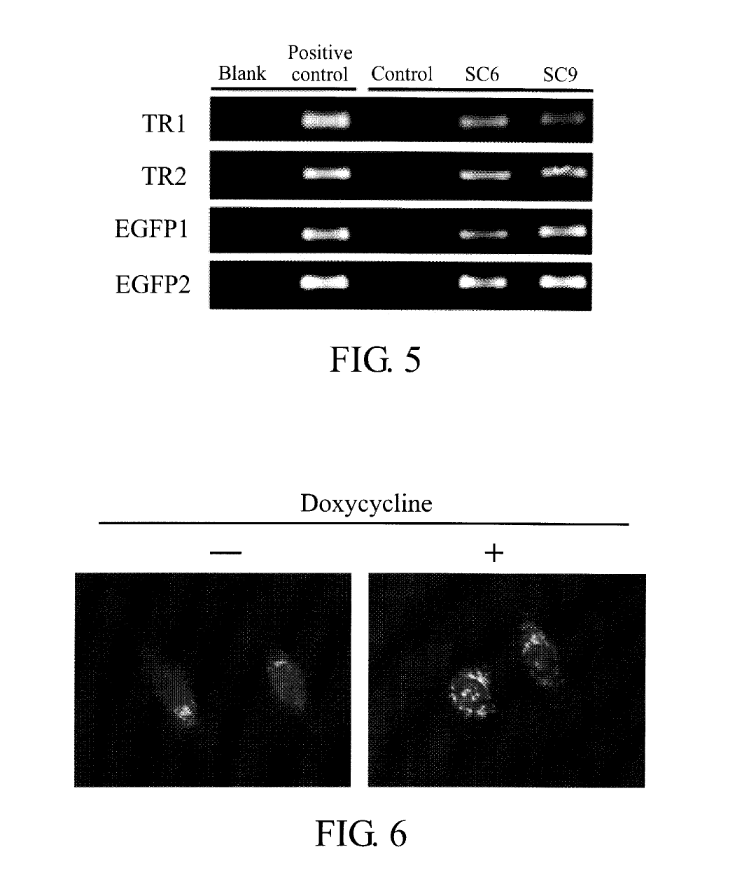

FIG. 5 shows the agarose gel electrophoresis results of the PCR products as obtained from PCR experiments using the genomic DNAs of SC6 and SC9 cells as templates and four different primer pairs (TR1, TR2, EGFP1 and EGFP2) designed based on the nucleotide sequences of recombinant plasmids pTrip10-TR and pTetO-EGFP, respectively. As can be clearly seen from FIG. 5, when using the genomic DNAs of the SC6 and SC9 cells as templates, PCR products amplified from PCR using the aforesaid four primer pairs were successfully obtained, indicating that the recombinant plasmids pTrip10-TR and pTetO-EGFP were indeed incorporated in the genomic DNAs of the obtained stable clones SC6 and SC9 cells.

FIG. 6 shows the expression of EGFP in SC6 cells as cultivated in the screening medium with (+) or without (−) the addition of 1 μg/mL doxycycline (400× magnification). Referring to FIG. 6, as compared to the control group, the SC6 cells cultivated in the presence of doxycycline were able to express EGFP. Similar experimental results were also observed in SC9 cells (data not shown). The applicants thus concluded that both the SC6 cells and the SC9 cells as obtained according to this invention were stable clones. In the following examples, the SC6 cells were selected for conducing experiments.

### Example 4

### Evaluation of the SC6 Cells for Use in Reporting the Inactivation of the Trip10 Promoter

### 1. Preparation of Methylated DNA Comprising Human Trip 10 Promoter

The recombinant plasmid pTrip10-yT&A as obtained in the section, entitled “1. Construction of recombinant plasmid pTrip10-yT&A,” of the above Example 1 was used as a template in a PCR experiment using the same primer pair and reaction conditions as set forth in Table 1 of said section. After completion of PCR, a DNA containing the human Trip10 promoter was purified and recovered via agarose gel electrophoresis, followed by extraction using the Clean and Gel Extraction Kit. The recovered DNA was subsequently quantitatively analyzed by NanoDrop (Thermo Scientific).

4 μg of the DNA containing the human Trip10 promoter was methylated by incubation with 20 U CpG methyltransferase (M.SssI) (New England BioLabs, Cat. No. M0226L) in the presence of 4 μL of 160 μM S-adenosylmethionine (SAM) (New England BioLabs, Cat. No. M0226L) at 37° C. for 4 hours, followed by heating at 65° C. for 5 minutes. Thereafter, the methylated DNA comprising the human Trip10 promoter (abbreviated as me_Trip10 DNA, infra) was heated at 95° C. for 5 minutes, followed by placing on ice for over 5 minutes, thereby yielding a single-stranded me_Trip10 DNA.

2. Transfection of SC6 Cells with me_Trip10 DNA

In a preparatory experiment, the applicants found that methylation of the Trip 10 promoter in the SC6 cells could be induced when using 0.4 μg of the single-stranded me_Trip10 DNA to transfect the SC6 cells, followed by continuous cell cultivation for 72 hours (data not shown). Therefore, in this example, the applicants employed 0.4 μg of the single-stranded me_Trip10 DNA to perform the experiments.

The SC6 cells as obtained in the above Example 3 were transfected with 0.4 μg of the single-stranded me_Trip10 DNA according to the procedures as set forth in the preceding section, entitled “2. Transfection,” of the General Experimental Procedures, and the transfection treatment was repeated at two day intervals thrice. Thereafter, the first time at which the liquid in each well was added to reach 1 mL was defined as Hour 0. Expression of EGFP in the SC6 cells having been transfected with the single-stranded me_Trip10 DNA was observed using an inverted fluorescence microscope at 0 hr, 24 hrs, 72 hrs and 120 hrs, respectively, and photographs were recorded using a digital camera connected to the inverted fluorescence microscope.

The obtained photographs were analyzed via an image analyzing software Scion Image. Briefly, the photographs were divided into several regions by the Scion Image software, in which the EGFP signal in a region visually observed as a black region was defined as 0, and the EGFP signal in a region that was visually observed to have the strongest fluorescence was defined as 255. Thereafter, the EGFP intensity was obtained by summing up the EGFP signals in all the regions, followed by dividing by the total area of said regions. Finally, the EGFP intensity at 0 hr was defined as 100%, and the EFGP intensity percentages of the EGFP intensities at other time points relative to that at 0 hr were calculated.

In order to verify that targeted DNA methylation of the Trip10 promoter occurred in the genomic DNAs of the SC6 cells, two days after the third transfection, a portion of the cells were harvested for use in the following semi-quantitative methylation-specific PCR (qMSP) and bisulfite sequencing.

3. Semi-Quantitative Methylation-Specific PCR (qMSP)

The qMSP experiment was conducted according to the procedures as set forth in P. S. Yan et al. (2006), Clin. Cancer Res., 64:6626-6636.

First, the genomic DNAs of the SC6 cells were purified using the Genomic DNA Purification Kit. 0.5 μg of the purified genomic DNAs were bisulfite converted using an EZ DNA Methylation kit (Zymo Research, #D5001) according to the manufacturer's instructions. Same experiments using the CpGenome Universal Methylated DNA (Chemicon, Cat. No. S7821), the genomic DNAs of the MCF7 cells, and the genomic DNAs of the SC6 cells that were transfected with the non-methylated DNA containing the human Trip10 promoter as obtained in the preceding section, entitled “Preparation of methylated DNA comprising human Trip 10 promoter,” were performed to serve as the positive control group, the negative control group and the mock-transfection control group, respectively.

The qMSP experiment was performed in 25 μL of a reaction mixture containing 4 μL of a template (bisulfite converted genomic DNA, 0.02 μg/μL), 2 μL of a primer pair (2.5 μM), 12.5 μL of 2× reaction buffer (SYBR Green Realtime PCR Master Mix, Toyobo, Cat. No. QPK201), and 6.5 μL ddH₂O. The primers used in the qMSP experiment are shown in Table 4, in which the primer pairs BR_{—}137 and BR_{—}138 listed therein were designed based on the human collagen type II alpha 1 gene (COL2A1 gene) and were used to amplify the serially diluted (1/10, 1/100 and 1/1000) bisulfite-converted CpGenome Universal Methylated DNA, respectively, so as to generate a quantification standard curve and to normalize the amounts of the methylated DNAs contained in the test samples.

TABLE 4
Primers used in the qMSP experiment

| Gene | Primer | Sequence (5′→3′) |
|---|---|---|
| Trip10 | h_Trip10_L_M1 | gagaggacgagagggatttc |
| | | (SEQ ID NO: 11) |
| | h_Trip10_R_M1 | ccaaacactacgattcgaaaac |
| | | (SEQ ID NO: 12) |
| Trip10 | h_Trip10_L_M2 | gaagtttatttgggagttttagtacgt |
| | | (SEQ ID NO: 13) |
| | h_Trip10_R_M2 | aataacctctctcaaccgcc |
| | | (SEQ ID NO: 14) |
| Col2A1 | BR_137 | tctaacaattataaactccaaccaccaa |
| | | (SEQ ID NO: 15) |
| | BR_138 | gggaagatgggatagaagggaatat |
| | | (SEQ ID NO: 16) |

The qMSP experiment was performed on the Bio-Rad iQ5 real time PCR machine, starting with a 5 min denaturation at 95° C., followed by running 40 cycles of the following reactions: holding at 95° C. for 30 sec and holding at 60° C. for 1 min. Concerning the melting curve validation, PCR was performed under the following conditions: holding at 55° C. for 1 min and 30 sec, followed by ramping up to 95° C. (0.5° C./30 sec). The presence of a single PCR product was confirmed by the appearance of a single melting peak.

The methylation percentage was calculated using the following equation:

A=(B/C)×100

A: Methylation percentage (%)

B: Intensity amplified by the Trip10 primer pair

C: Intensity amplified by the Col2A1 primer pair

### 4. Bisulfite Sequencing:

0.02 μg of the bisulfite converted genomic DNA of the SC6 cells as obtained above was used as the template. PCR was conducted in 25 μL of a reaction mixture containing 4 μL of the bisulfite converted genomic DNA, 4 μL of a primer pair (2.5 μM), 1 μL of dNTPs (10 mM), 5 μL of MgCl₂ (50 mM), 2.5 μL of 10× Taq buffer, 0.2 μL of 1.25 U Taq DNA polymerase (Fermentus, Cat. No. EP0402), and 8.3 μL ddH₂O. The primer pair as used in the PCR is shown below:

h_bis_Trip10_TR_F2 primer:

| | |
|---|---|
| 5′-gggaaaggggaaaaggagatggg-3′ | (SEQ ID NO: 17) |
| h_bis_Trip10_TR_R2 primer: | |
| 5′-atcttaccaactttccccttctaaaaaac-3′ | (SEQ ID NO: 18) |

The PCR amplification was performed on a Mastercycler PCR machine (Eppendorf), starting with a 5 min denaturation at 95° C., followed by running 37 cycles of the following reactions: denaturation at 95° C. for 30 sec; primer annealing at 60° C. for 30 sec; and elongation at 72° C. for 1 min and 15 sec; and finally, an extra extension at 72° C. for 4 min. In the meantime, same experiment using the genomic DNA of the SC6 cells transfected with a non-methylated DNA containing the human Trip10 promoter as obtained in the preceding section, entitled “Preparation of methylated DNA comprising human Trip 10 promoter,” was performed to serve as the mock-transfection control group.

After completion of PCR, the PCR products were verified by agarose gel electrophoresis, followed by purification and recovery using the Clean and Gel Extraction Kit. The recovered PCR products were subcloned using the yT&A Cloning Vector Kit according to the procedures as set forth in the preceding section, entitled “1. Construction of recombinant plasmid pTrip10-yT&A,” of the above Example 1. The plasmid DNAs of 10˜15 insert-positive clones were extracted by a High-Speed Plasmid Mini Kit, followed by cleavage using restriction enzymes EcoRI and BamHI and agarose gel electrophoresis, so as to verify the molecular weights thereof. The verified plasmid DNAs were subjected to a sequencing analysis conducted by Tri-I Biotech, Inc.

### Results:

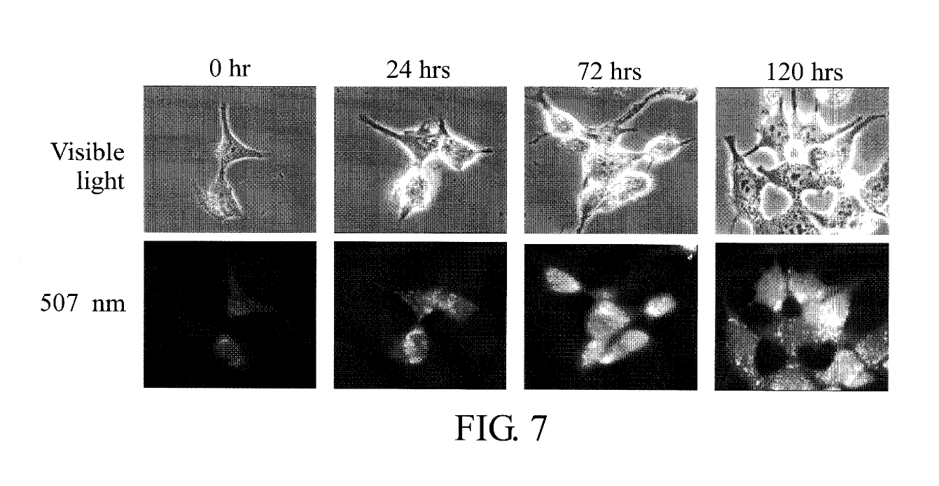

FIG. 7 shows the expression of EGFP in SC6 cells having been transfected with single-stranded me_Trip10 DNA as observed at different time points (0 hr, 24 hrs, 72 hrs and 120 hrs) under visible light (the four upper panels) or at a wavelength of 507 nm (the four lower panels) (400× magnification).

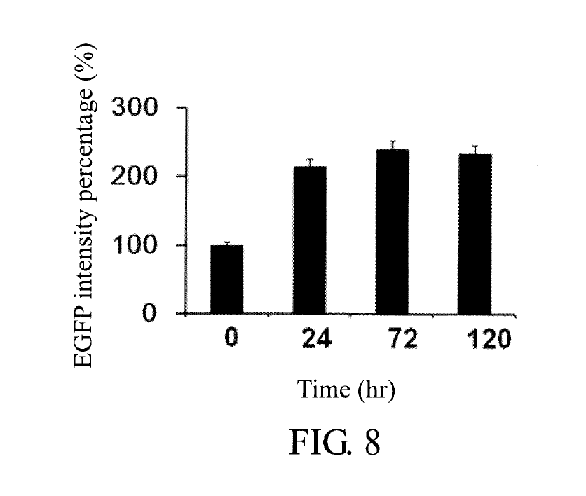

FIG. 8 is a bar diagram showing the EGFP intensity percentages of the observed EGFP expressions in the four lower panels of FIG. 7 as quantitatively analyzed by an image analyzing software.

Referring to FIGS. 7 and 8, when the SC6 cells were transfected with single-stranded me_Trip10 DNA, expression of EGFP in the SC6 cells increased gradually with the increased times of transfection. The phenomenon was obviously observed after several passages (i.e., Hour 72-Hour 120). This experiment shows that the SC6 cells are able to continuously and instantaneously reveal the methylation patterns of the Trip10 promoter via detecting the EGFP expression thereof.

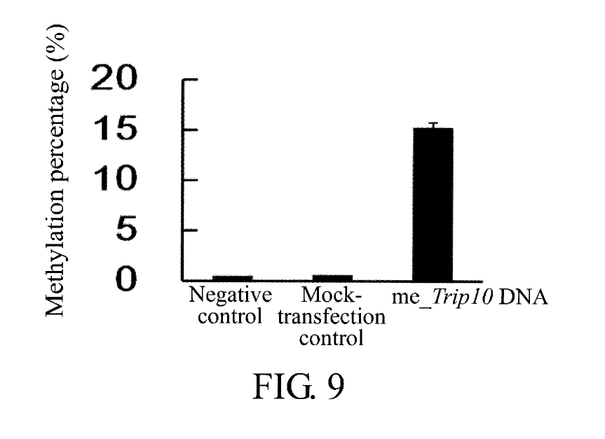

FIG. 9 is a bar diagram that shows the methylation percentages of the Trip10 promoter and the first exon region in SC6 cells having been transfected with single-stranded me_Trip10 DNA. Referring to FIG. 9, the methylation percentage of the Trip10 promoter and the first exon region in the SC6 cells having been transfected with single-stranded me_Trip10 DNA is higher than the negative control group and the mock-transfection control group by about 15%.

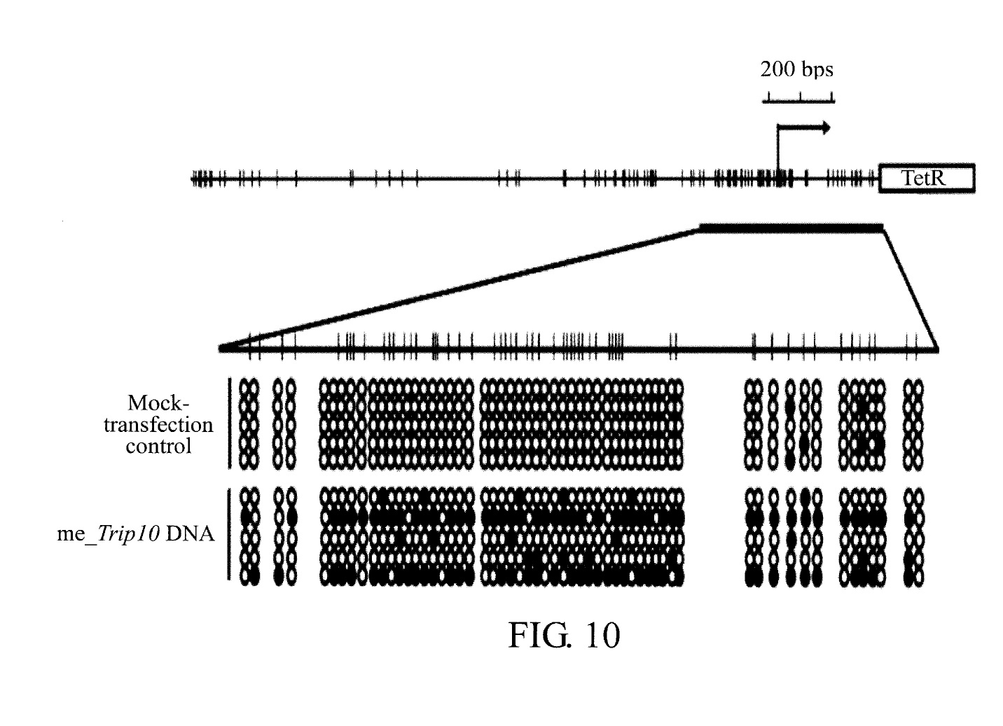

In the bisulfite sequencing experiment, in order to prevent false positive amplifications caused by the endogenous Trip10 promoter, the applicants designed a specific primer pair Bis (see FIG. 4) and used the same to run PCR. FIG. 10 shows the bisulfite sequencing results of the Trip 10 promoter and the first exon region in SC6 cells having been transfected with single-stranded me_Trip10 DNA. Referring to FIG. 10, most of the CpG dinucleotides within the Trip10 promoter and the first exon region in the cells of the mock-transfection control group are non-methylated (white circles represent non-methylated CpG dinucleotides). After introduction of single-stranded me_Trip10 DNA, endogenous DNA methylation (black circles represent methylated CpG dinucleotides) was observed in the SC6 cells having been transfected with the single-stranded me_Trip10 DNA.

In conclusion, the SC6 cells established according to this invention are able to visually report targeted DNA methylation patterns via detecting the EGFP expression thereof.

In a previous study, the applicants found from a ChIP-on-chip analysis that ERα acted as a ligand-inducible transcription factor and it was capable of regulating the transcription of downstream target gene (e.g., Trip10) by binding to the promoter region of a downstream target gene (e.g., Trip10) or tethering to other transcription factors. It was further indicated in said study that in order to reactivate silenced locus (i.e., to restore transcription activity), ERα and DNA demethylation are required (Y. W. Leu et al., (2004), Cancer Research, 64:8184-8192). Therefore, in order to replay the process of reactivating the Trip10 promoter, in the following example, the applicants provided an inactivated Trip10 promoter in the SC6 cells so as to mimic an abnormal DNA methyl modification. Thereafter, the applicants added into the growth medium (a) 17β-estradiol (an estrogen analogoue) and (b) 17β-estradiol and 5-aza-2′-deoxycytidine (a demethylating agent), respectively, followed by observation of the EGFP expression, so as to understand the influence of the 17β-estradiol and 5-aza-2′-deoxycytidine upon the transcription activity of the Trip10 promoter.

### Example 5

### Evaluation of SC6 Cells for Use in Reporting the Change of the Transcription Activity of Trip10 Promoter

### I. Effect of 17β-Estradiol Upon the Trip10 Promoter Against the DNA Methylation Thereof.

### Experimental Procedure:

First, a proper amount of 17β-estradiol was dissolved in alcohol to form a stock solution having a concentration of 10 mg/mL. The SC6 cells as obtained in the above Example 3 were plated into each well of 6-well plates (1×10⁵ cells/2 mL screening medium/well), followed by cultivation overnight in an incubator (37° C., 95% O₂/5% CO₂) to permit cell attachment. Thereafter, a proper amount of the stock solution as prepared above was added into each well so that 17β-estradiol contained in each well had a final concentration of 10 ng/mL. After cultivation at 37° C. for 24 hours, the SC6 cells were transfected with 0.4 μg of single-stranded me_Trip10 DNA according to the transfection treatment as described in the section, entitled “2. Transfection of SC6 cells with me_Trip10 DNA” of the above Example 4, except that the growth medium was supplemented with 10 ng/mL 17β-estradiol and was added into each well until a final volume of 1 mL was reached.

The first time at which the liquid in each well was added to reach 1 mL was defined as Hour 0. Expression of EGFP in the SC6 cells was observed using an inverted fluorescence microscope at 0 hr, 24 hrs, 72 hrs and 120 hrs, respectively, and photographs were recorded using a digital camera connected to the inverted fluorescence microscope.

### Results:

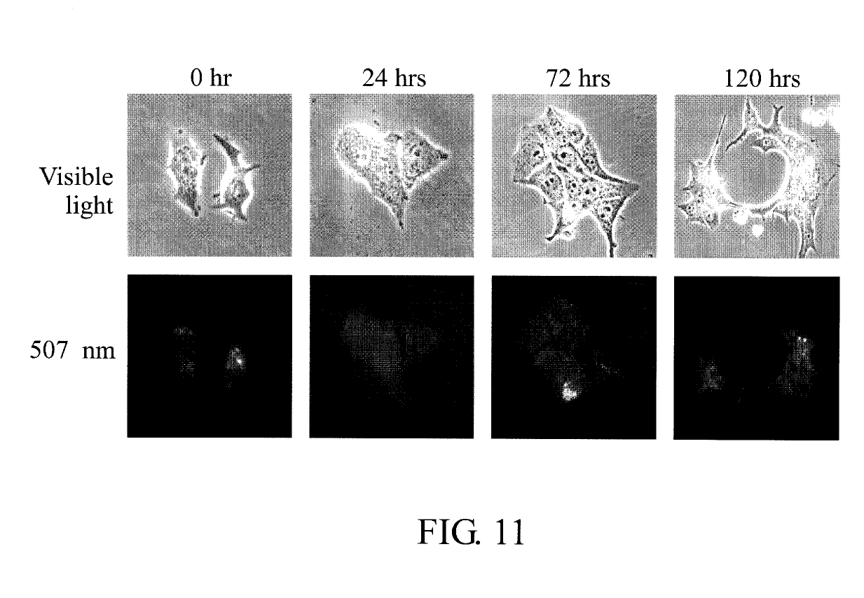

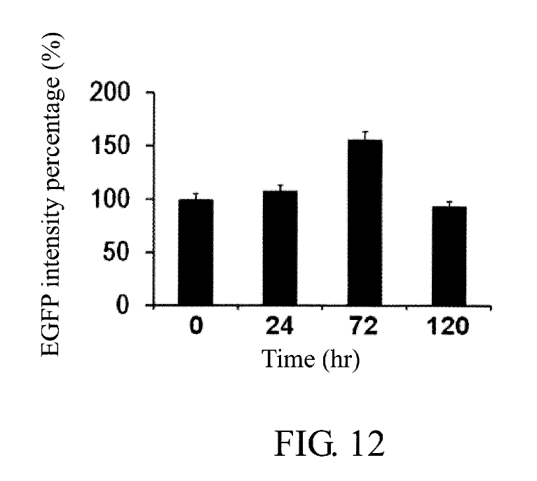

FIG. 11 shows the expression of EGFP in SC6 cells having been transfected with single-stranded me_Trip10 DNA as observed at different time points (0 hr, 24 hrs, 72 hrs and 120 hrs) under visible light (the four upper panels) or at a wavelength of 507 nm (the four lower panels) (400× magnification). FIG. 12 is a bar diagram showing the EGFP intensity percentages of the observed EGFP expressions in the four lower panels of FIG. 11 as quantitatively analyzed by an image analyzing software. Referring to FIGS. 11 and 12, as compared to the EGFP intensity at 0 hr, except that the EGFP intensity at 72 hrs increased by about 150%, the EGFP intensities at other time points were around 100%. This experiment reveals that if the SC6 cells were cultivated in medium containing 17β-estradiol before, during and after transfection with the single-stranded me_Trip10 DNA, the Trip10 promoter could be prevented from methylation as induced by me_Trip10 DNA and remained non-methylated.

### II. Effect of Co-Addition of 5-Aza-2′-Deoxycytidine and 17β-Estradiol Upon the Reactivation of Inactivated Trip10 Promoter

The transfection-treated SC6 cells, which were harvested two days after the third time of transfection according to the procedures set forth in the section, entitled “2. Transfection of SC6 cells with me_Trip10 DNA,” of the above Example 4, were plated into each well of 6-well plates (1×10⁴ cells/2 mL screening medium/well), followed by cultivation overnight in an incubator (37° C., 95% O₂/5% CO₂) to permit cell attachment. 10 mM 5-aza-2′-deoxycytidine (dissolved in DMSO) was subsequently added into each well to reach a final concentration of 25 μM (Day 0). Thereafter, medium changes were performed every two days with fresh screening medium supplemented with 25 μM 5-aza-2′-deoxycytidine. From the second time of medium change (Day 4), the screening medium was further supplemented with 10 ng/mL 17β-estradiol. Expression of EGFP in the SC6 cells was observed using an inverted fluorescence microscope prior to making the medium change, and photographs were recorded using a digital camera connected to the inverted fluorescence microscope.

### Results:

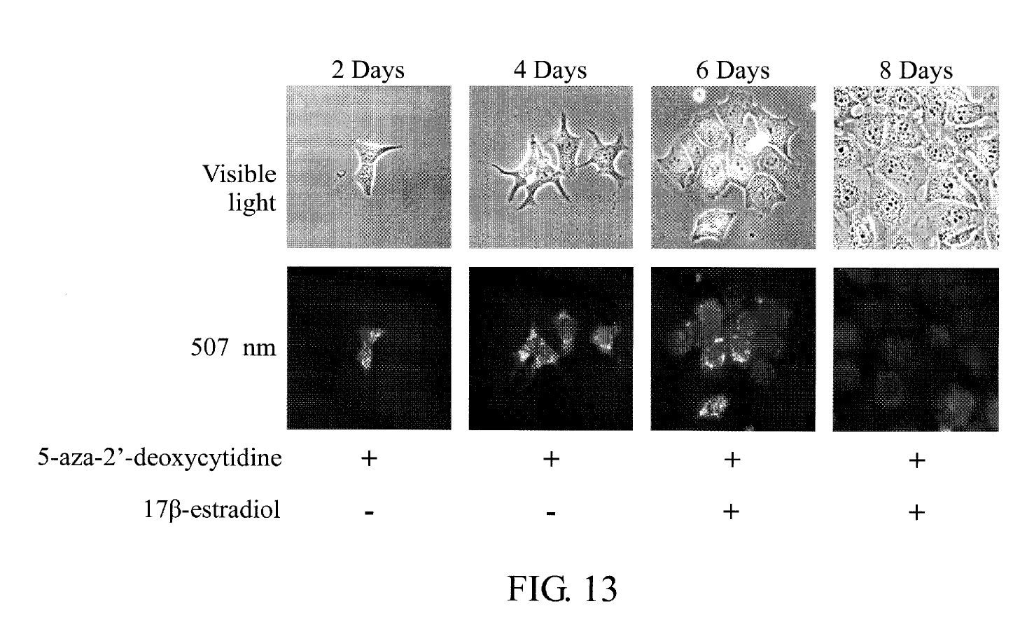

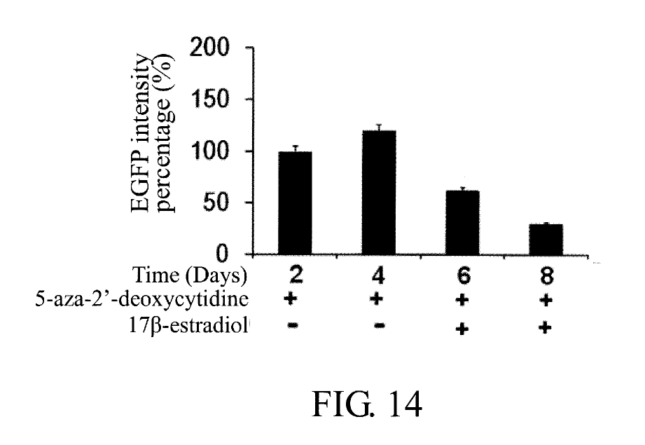

FIG. 13 shows the expression of EGFP in SC6 cells having been transfected with single-stranded me_Trip10 DNA as observed at different time points (2 days, 4 days, 6 days and 8 days) under visible light (the four upper panels) or at a wavelength of 507 nm (the four lower panels) (400× magnification). FIG. 14 is a bar diagram showing the EGFP intensity percentages of the observed EGFP expressions in the four lower panels of FIG. 13 as quantitatively analyzed by an image analyzing software. Referring to FIGS. 13 and 14, as compared to the EGFP intensity (100%) at Day 2, the EGFP intensity at Day 4 could remain over 100%. However, the EGFP intensity at Day 6 (cells being cultivated in the presence of 5-aza-2′-deoxycytidine and 17β-estradiol for two days) decreased to about 50%, and the EGFP intensity at Day 8 (cells being cultivated in the presence of 5-aza-2′-deoxycytidine and 17β-estradiol for four days) decreased to about 25%. This experiment reveals that with respect to the Trip10 promoter, the addition of 5-aza-2′-deoxycytidine alone is insufficient to reactivate the Trip10 promoter, and only the co-addition of 5-aza-2′-deoxycytidine and 17β-estradiol can reverse the methylation of the Trip10 promoter, thereby reactivating the Trip10 promoter to restore its transcription activity. This finding is consistent with the applicants' previous research results (Y. W. Leu et al. (2004), supra)

On the other hand, the experimental results reveal that SC6 cells can continuously grow with time in the screening medium supplemented with 5-aza-2′-deoxycytidine. This indicates that 5-aza-2′-deoxycytidine does not cause damage to the SC6 cells and, hence, has no cytotoxicity.

In conclusion, the applicants have developed a new method for screening a candidate compound as a demethylating agent. This method can instantaneously reflect the effect of the candidate compound upon the DNA methylation pattern of an interested promoter in living cells by detecting the expression of a reporter gene, so that whether or not the candidate compound is suitable to act as a demethylating agent can be determined. Meanwhile, the method according to this invention has the advantages that there is no need to destroy the test cells, thereby enabling the researchers and investigators to conveniently and continuously observe the test cells during the screening process so as to determine the cytotoxicity of the candidate compound. Therefore, it is contemplated that the screening method according to this invention can be used to extensively and efficiently screen demethylating agents.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. A three-component gene expression reporting system for mammalian cells, the system comprising:
a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene;
a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and
a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
wherein introduction of the methylated polynucleotide into a mammalian cell that has been co-transfected by the first and second expression cassettes results in the methylation of the one or more CpG islands of the second promoter sequence in the co-transfected mammalian cell and progeny cell thereof, thereby repressing the first nucleic acid sequence to express the first gene product.

2. The three-component gene expression reporting system for mammalian cells as claimed in claim 1, wherein both the first operator region and the first nucleic acid sequence are heterologous to the mammalian cell.

3. The three-component gene expression reporting system for mammalian cells as claimed in claim 1, wherein both the first operator region and the first nucleic acid sequence are derived from a gene of a microbial cell.

4. The three-component gene expression reporting system for mammalian cells as claimed in claim 1, wherein the first operator region comprises a tetracycline operator, and the first gene product encoded by the first nucleic acid sequence is a tetracycline repressor.

5. The three-component gene expression reporting system for mammalian cells as claimed in claim 1, wherein the first operator region comprises a Lac operator, and the first gene product encoded by the first nucleic acid sequence is a Lac repressor.

6. The three-component gene expression reporting system for mammalian cells as claimed in claim 1, wherein the first operator region comprises a GUS operator, and the first gene product encoded by the first nucleic acid sequence is a GUS repressor.

7. The three-component gene expression reporting system for mammalian cells as claimed in claim 1, wherein the first promoter sequence comprises a promoter selected from the group consisting of a CMV promoter, a SV40 initial promoter, a RSV-promoter, a HSV-TK promoter, a U6 promoter, a CMV-HSV thymidine kinase promoter, a SRα promoter and a HIV•LTR promoter.

8. The three-component gene expression reporting system for mammalian cells as claimed in claim 1, wherein the reporter gene encodes a reporter gene product selected from the group consisting of: green fluorescent protein, enhanced green fluorescent protein, red fluorescent protein, yellow fluorescent protein, blue fluorescent protein, td-Tomato, mCherry, firefly luciferase, Renilla luciferase, β-galactosidase, β-glucuronidase, and a fusion protein comprising one or more of the foregoing proteins.

9. The three-component gene expression reporting system for mammalian cells as claimed in claim 1, wherein the second promoter sequence comprises a promoter selected from the group consisting of a Trip10 promoter, a Casp8AP2 promoter, a ENSA promoter, and a H1C1 promoter.

10. The three-component gene expression reporting system for mammalian cells as claimed in claim 1, wherein the methylated polynucleotide has a length ranging from 22 to 2,000 nucleotides.

11. The three-component gene expression reporting system for mammalian cells as claimed in claim 1, wherein the first expression cassette further comprises a first marker gene and the second expression cassette further comprises a second marker gene, wherein the first marker gene, the second marker gene and the reporter gene differ from each other, so that a mammalian cell co-transfected by the first and second expression cassettes can be selected from a screening test.

12. The three-component gene expression reporting system for mammalian cells as claimed in claim 11, wherein the first marker gene and the second marker gene are independently selected from the group consisting of a hygromycin resistance gene, a neomycin resistance gene, a gentamycin resistance gene, a blasticidin resistance gene, a zeocin resistance gene, and a puromycin resistance gene.

13. A kit for screening a demethylating agent, comprising:
(a) a recombinant mammalian cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product; and
(b) a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
wherein introduction of the methylated polynucleotide into the recombinant mammalian cell results in the methylation of the one or more CpG islands of the second promoter sequence in the recombinant mammalian cell and progeny cell thereof, thereby repressing the first nucleic acid sequence to express the first gene product.

14. The kit of claim 13, wherein the recombinant mammalian cell is obtained by co-transfecting a human cell with the first and second expression cassettes.

15. A method for screening a candidate compound as a demethylating agent, comprising:
providing a first population of a recombinant mammalian cell, each cell comprising:
(i) a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
(ii) a second expression cassette, which comprises: a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product;
introducing into the first population of the recombinant mammalian cell a methylated polynucleotide selected from the group consisting of:
(i) a single-stranded molecule, which has a nucleotide sequence identical to or fully complementary to that of a portion of the second promoter sequence;
(ii) a double-stranded molecule, one strand of which has a nucleotide sequence identical to or fully complementary to that of a portion of the second promoter sequence; and
(iii) a combination of (i) and (ii);
so that the one or more CpG islands of the second promoter sequence in the first population of the recombinant mammalian cell or progeny cell thereof become methylated, thereby repressing the first nucleic acid sequence to express the first gene product;
cultivating the first population of the recombinant mammalian cell for a period of time to obtain a second population of the recombinant mammalian cell;
detecting the second population of the recombinant mammalian cell to obtain a first expression level of the reporter gene;
treating the second population of the recombinant mammalian cell with a candidate compound, followed by cultivation for a period of time, so as to obtain a third population of the recombinant mammalian cell; and
detecting the third population of the recombinant mammalian cell to obtain a second expression level of the reporter gene,
wherein the candidate compound is deemed as a demethylating agent if the obtained second expression level of the reporter gene is lower than the obtained first expression level of the reporter gene.

16. The method of claim 15, wherein detecting the recombinant mammalian cell is implemented by colormetry, fluorimetry, luminescent analysis, enzyme linked immunoSorbent assay or flow cytometry.

17. The method of claim 15, wherein when treating the second population of the recombinant mammalian cell with the candidate compound, a reagent that assists in reversion of the methylation of the one or more CpG islands of the second promoter sequence is simultaneously applied to the second population of the recombinant mammalian cell.

18. The method of claim 17, wherein the second promoter sequence comprises a Trip10 promoter, and the reagent is an estrogen.

19. A recombinant mammalian cell comprising:
a first expression cassette, which comprises in sequence along a transcription direction: a first promoter sequence operable in a mammalian cell, a first operator region operable in the mammalian cell, and a reporter gene, wherein the first promoter sequence and the first operator region control the expression of the reporter gene; and
a second expression cassette, which comprises a second promoter sequence operable in the mammalian cell, and a first nucleic acid sequence located downstream of the second promoter sequence and encoding a first gene product capable of binding to the first operator region to repress the expression of the reporter gene, wherein the second promoter sequence has one or more CpG islands in the sequence thereof and controls the first nucleic acid sequence to express the first gene product,
wherein in the recombinant mammalian cell, the one or more CpG islands in the second promoter sequence have been methylated to result in repression of the first nucleic acid sequence, thereby allowing the reporter gene to be expressed in the recombinant mammalian cell.

20. A method for screening a candidate compound as a demethylating agent, comprising:
providing a first population of a recombinant mammalian cell as claimed in claim 19;
detecting the first population of the recombinant mammalian cell to obtain a first expression level of the reporter gene;
treating the first population of the recombinant mammalian cell with a candidate compound, followed by cultivation for a period of time, so as to obtain a second population of the recombinant mammalian cell; and
detecting the second population of the recombinant mammalian cell to obtain a second expression level of the reporter gene,
wherein the candidate compound is deemed as a demethylating agent if the obtained second expression level of the reporter gene is lower than the obtained first expression level of the reporter gene.
